(19) United States
(12) Patent Application Publication
(10) Pub. No.: US 2006/0142187 A1
(43) Pub. Date: Jun. 29, 2006

(54) Methods for modulating cell-to-cell adhesion using an agonist of C1INH-type protein activity

(75) Inventors: Alvin E. Davis, Boston, MA (US); Shenghe Cai, Quincy, MA (US)

Correspondence Address: LAHIVE & COCKFIELD, 28 STATE STREET, BOSTON, MA, 02109, US

(73) Assignee: CBR Institute for Biomedical Research, Inc., Boston, MA (US)

(21) Appl. No.: 11/274,009

(22) Filed: Nov. 14, 2005

Related U.S. Application Data
(63) Continuation of application No. PCT/US04/15445, filed on May 17, 2004.
(60) Provisional application No. 60/471,044, filed on May 15, 2003.
(60) Provisional application No. 60/471,122, filed on May 16, 2003.

(51) Int. Cl. A61K 38/17

(52) U.S. Cl. 514/8

(57) ABSTRACT

The present invention is based, at least in part, on the discovery that plasma C1INH contains a sialyl Lewis^{x} related moiety on its N-glycan and is capable of binding selectin molecules. The invention provides methods for modulating cell-to-cell adhesion or cell migration comprising contacting a cell with a C1INH-type protein or fragment thereof or a nucleic acid encoding a C1INH-type protein or fragment thereof, such that cell-to-cell adhesion is modulated. The invention also provides methods for treating or preventing cell adhesion related disorders in a subject comprising administering to the subject an effective amount of a C1INH-type protein or fragment thereof or a nucleic acid encoding a C1INH-type protein or fragment thereof.

### RELATED APPLICATIONS

This application is a continuation of PCT/US2004/015445, filed May 17, 2004, which claims the benefit of prior-filed provisional patent application Ser. No. 60/471,044, filed May 15, 2003, and provisional patent application Ser. No. 60/471,122, filed May 16, 2003. The entire content of both the above-referenced applications are incorporated herein by this reference.

### GOVERNMENT RIGHTS

This invention was made at least in part with government support under grant no. HD22082 and grant no. HD33727 awarded by the National Institutes of Health. The government has certain rights to this invention.

### BACKGROUND OF THE INVENTION

The ability of cells to adhere to one another plays a critical role in embryonic development as well as normal and disease or pathological processes. Cell adherence is mediated by cell adhesion molecules which are generally glycoproteins and expressed on the cell surface. Several classes of adhesion molecules have been identified and include the members of the immunoglobulin (Ig) superfamily, the integrins and the selecting.

Thus far three human selectin proteins have been identified, E-selectin (formerly ELAM-1), L-selectin (formerly LAM-1) and P-selectin (formerly PADGEM or GMP-140). The selectin proteins are characterized by a N-terminal lectin-like domain, an epidermal growth factor-like domain, and regions of homology to complement binding proteins. E-selectin is induced on endothelial cells several hours after activation by cytokines, mediating the calcium-dependent interaction between neutrophils and the endothelium. L-selectin is the lymphocyte homing receptor, and P-selectin rapidly appears on the cell surface of platelets when they are activated, mediating calcium-dependent adhesion of neutrophils or monocytes to platelets. P-selectin is also found in the Weibel-Palade bodies of endothelial cells; upon its release from these vesicles P-selectin mediates early binding of neutrophils to histamine-or thrombin-stimulated endothelium.

Selectins are believed to mediate adhesion through specific interactions with ligands present on the surface of target cells, e.g., platelets and leukocytes. Generally the ligands of selectins are comprised, at least in part, of a carbohydrate moiety (e.g., sialyl Lewis^{x} (sLe^{x}) and sialyl Lewis^{a} (sLe^{a})). Although many different putative ligands of selectins have been described, their physiological relevance has not been elucidated in all cases. Selectins and some of their counter-receptors function also as signal-transducing receptors, significantly contributing to leukocyte and endothelial cell activation.

Leukocyte recruitment, e.g., capture of blood-borne leukocytes onto vascular endothelium, proceeds via a two-step mechanism, with each step mediated by a distinct receptor-ligand pair. Selectins have been implicated in mediating interactions between endothelial cells and leukocytes in what is known as “leukocyte rolling”. Cells first transiently adhere, or “roll” (via interactions between selectins and sialyl-Lewis^{x}), and then firmly adhere to the vascular wall (via interactions between integrins and ICAM-1), which is generally believed to be the prerequisite for firm adhesion and subsequent transendothelial migration of leukocytes into tissues (Moore, K. L. (1998) Leuk Lymphoma 29:1-15) and is an essential component of the immune response. Soluble P-selectin has also been shown to be shed from both activated platelets and endothelium attenuating effect on inflammatory progression. Additionally, such selectin-mediated cellular adhesion also occurs in thrombotic disorders and parasitic diseases and may be implicated in metastatic spread of tumor cells. P-selectin rapidly appears on the cell surface of platelets when they are activated, mediating calcium-dependent adhesion of neutrophils or monocytes to platelets.

C1 inhibitor (C1INH), a member of the serpin (serine proteinase inhibitor) family, regulates all three pathways of complement activation. It is the sole natural inhibitor of C1r and C1s, is an inhibitor of the lectin pathway via inactivation, of mannan binding lectin associated serine proteinase-1 and 2, and inhibits the alternative pathway of activation by binding to C3b (Jiang, H. et al. (2001) J Exp Med 194:1609-1616). It is also the major regulator of coagulation factors XI and XII, and of plasma kallikrein. Therefore, C1INH is an inhibitory protein in the complement system, the contact system of kinin generation, and the intrinsic coagulation pathway.

C1INH is the most heavily glycosylated plasma protein (Davis III, A. E. (1988) Ann Rev Immunol 6:595-628). Of its 104 KD apparent molecular mass, the protein moiety of 478 amino acids accounts for only 52,869 Daltons. Carbohydrate, therefore, contributes about 35% of the total molecular mass (Bock, S. C. (1986) Biochemistry 25:4292-4301; Harrison, R. A. (1983) Biochemistry 22:5001-5007; Perkins, S. J. et al. (1990) J. Mol Biol 214:751-763). C1INH contains 13 definitively identified glycosylation sites (7 O-linked and 6 N-linked), as well as an additional 7 potential O-linked glycosylation sites. Ten of the 13 glycosylation sites are located in the amino terminal domain (first 100 residues), which is the longest amino terminal extension among the known serpins.

The role of carbohydrate in the function of C1INH remains unknown, although it may contribute to its clearance from plasma (Minta, J. O. (1981) J. Immunol 126(1):254-249). Although it has been suggested that carbohydrate may contribute to conformational stability and binding kinetics toward target proteases (Bos, I. G., et al. (2002) Immunobiol 205(4-5):518-533), the data previously available indicated that carbohydrate does not play a major role in inhibitory activity (Coutinho, M. et al. (1994) J Immunol 153(8):3648-3654; Reboul, A. et al. (1987) Biochem J 244(1): 117-121).

### SUMMARY OF THE INVENTION

The present invention is based, at least in part, on a novel anti-inflammatory function of C1INH that is unrelated to its previously identified protease inhibitory activity. In one embodiment, the present invention is based on the discovery that plasma C1INH contains a specific glycoprotein, e.g., a sialyl-Lewis^{x} related moiety, on its N-glycan and specifically binds to selectin molecules, e.g., E-selectin, P-selectin, including soluble P-selectin, and L-selectin. The expression of selectin molecules on cells mediate the interaction, e.g., cell-to-cell adhesion of leukocytes and endothelial cells in a process known as “leukocyte rolling.” This process is required for subsequent firm binding of leukocytes to the endothelium lining, the vascular wall, and subsequent transendothelial migration of leukocytes, e.g., an immune response. The expression of selectin molecules on platelets or shedding of soluble P-selectin from activated platelets also mediates the interaction between platelets and leukocytes. Accordingly, a C1INH-type protein binds selectin molecules and thereby modulate cell-to-cell adhesion and treats or prevents cell adhesion related disorders.

Hence, one aspect of the invention provides a method for modulating cell-to-cell adhesion in a subject comprising administering to the subject an effective amount of a composition comprising an agonist of C1INH-type protein activity, e.g., a C1INH-type protein, or fragment thereof, such that cell-to-cell adhesion is modulated in the subject. Another aspect of the invention provides modulating cell-to-cell adhesion in a subject comprising administering to the subject a nucleic acid molecule encoding a C1INH type protein, or a fragment thereof.

A further aspect of the invention provides a method for treating or preventing cell adhesion related disorders in a subject comprising administering to the subject an effective amount of a composition comprising an agonist of C1INH-type protein activity, e.g., a C1INH-type protein, or fragment thereof, a nucleic acid molecule encoding a C1INH type protein, or a fragment thereof, thereby treating or preventing a cell adhesion related disorder in a subject. In one embodiment, the cell adhesion related disorder is selected from the group consisting of myocardial infarction, bacterial or viral infection, metastatic conditions, arthritis, gout, uveitis, acute respiratory distress syndrome, asthma, emphysema, delayed type hypersensitivity reaction, systemic lupus erythematosus, thermal injury such as burns or frostbite, autoimmune thyroiditis, experimental allergic encephalomyelitis, multiple sclerosis, multiple organ injury syndrome secondary to trauma, diabetes, Reynaud's syndrome, neutrophilic dermatosis (Sweet's syndrome), inflammatory bowel disease, Grave's disease, glomerulonephritis, gingivitis, periodontitis, hemolytic uremic syndrome, ulcerative colitis, Crohn's disease, necrotizing enterocolitis, granulocyte transfusion associated syndrome, cytokine-induced toxicity, fetal development, and thrombotic diseases.

One aspect of the present invention is based on a method for modulating cell-to-cell adhesion comprising contacting a cell with an agonist of C1INH-type protein activity, e.g., a C1INH-type protein, or fragment thereof, or a nucleic acid molecule encoding a C1INH type protein, or a fragment thereof, such that cell-to-cell adhesion is modulated. In one embodiment, a P-selectin expressing cell is contacted by C1INH-type protein and modulates cell-to-cell adhesion. In another embodiment, an E-selectin expressing cell is contacted by C1INH-type protein, fragment thereof, or a nucleic acid molecule encoding a C1INH type protein. In one embodiment the cell that expresses a selectin molecule may be a leukocyte, platelet, or endothelial cell. In a related embodiment, a C1INH-type protein binds to a selectin molecule, e.g., a soluble selectin molecule, to modulate cell-to-cell adhesion. In another embodiment, cell-to-cell adhesion is increased. In still another embodiment, cell-to-cell adhesion is decreased.

In one embodiment of the invention, the C1INH-type protein is C1INH. In another embodiment, the C1INH-type protein is an amino-terminal fragment of C1INH-type protein which retains its ability to bind to a selectin molecule. In another embodiment of the invention, the C1INH-type protein is a carboxy-terminal fragment of C1INH-type protein which retains its ability to bind to a selectin molecule. In another embodiment, C1INH-type protein specifically binds to a selectin molecule but does not inhibit activation of the complement system. In a further embodiment, C1INH-type protein specifically binds to a selectin molecule but does not inhibit activation of the contact system. In yet another embodiment, C1INH-type protein binds to a selectin molecule but lacks substantial protease inhibition activity.

The invention also encompasses processes for producing a C1INH-type protein comprising (a) co-transforming a host cell with a DNA encoding a C1INH-type proteins and a DNA encoding a fucosyltransferase capable of synthesizing sialyl Lewis X (sLe^{x}) or sialyl Lewis A (sLe^{a}) (such as an (α1,3/α1,4) fucosyltransferase or an (α1,3) fucosyltransferase), each of said DNAs being operably linked to an expression control sequence; (b) culturing the host cell in suitable culture medium; and (c) purifying the C1INH-type protein from the culture medium.

### BRIEF DESCRIPTION OF THE DRAWINGS

FIGS. 1A-C depict the reactivity of C1INH with the monoclonal antibodies HECA-452 and CSLEX1. A: Lanes 1-4 are plasma-derived C1INH, 8, 4, 2, and 1 μg, respectively. Lanes 5-8 are U937 lysate, LEC11 lysate, CHO-K1 lysate and BSA, respectively. B: Lane 1 is BSA (20 μg), lanes 2 and 3 are plasma-derived C1INH (5 and 2.5 μg, respectively), and lane 4 is U937 lysate. C: The recombinant C1INH expressed in LEC11 cells can be detected with HECA452 (lane 1) while that in CHO-K1 cells showed no signal (lane 2). The results of these Western Blots indicate that C1INH bears a sialyl Lewis^{x}-related moiety.

FIGS. 2A-B depict the presence of the sialyl Lewis^{x}-related moiety on C1INH. Deglycosylated C1INH was subjected to Western blot analysis with HECA-452 (A) and anti-C1INH antiserum (B). Lane 1 is untreated plasma-derived C1INH (5 μg), lane 2 is C1INH treated with N-Glycosidase F (5 μg), and lane 3 is C1INH treated with O-glycosidase and Neuraminidase (15 μg). The results of these Western Blots indicate that the sialyl Lewis^{x}-related moiety of C1INH is located on the N-glycan of C1INH.

FIGS. 3A-B depict the results of FACS analysis of plasma-derived C1INH demonstrating binding of C1INH to P- and E-selectin/IgG. (A) CHO/E (unshaded) compared with CHO-K1 (shaded). (B) CHO/P (unshaded) compared with CHO-K1 (shaded).

FIG. 4 depicts the results of co-immunoprecipitation of endothelial cell P and E-selectin with anti-C1INH antibody. The bound proteins were separated on SDS-PAGE and E-selectin (A) or P-selectin (B) was detected on the blot.

FIG. 5 depicts the effect of E-selectin on C1INH complex formation with C1s. Lane 1 is C1INH, lane 2 is C1s, lane 3 is E-selectin, lane 4 is C1INH incubated with C1s, and lane 5 is C1INH incubated with C1s in presence of E-selectin. These results demonstrate that E-selectin has no effect on C1INH complex formation with C1s.

FIG. 6 is a graph depicting the inhibition of U937-endothelial cell adhesion by C1INH.

FIG. 7 depicts the nucleotide and amino acid sequence of C1INH (SEQ ID NOs:1 and 2, respectively). The C1INH protein contains a signal peptide of 22 residues in the amino-terminal end which is cleaved resulting in a 478 amino acid mature protein. The amino terminal domain of C1INH contains 7 repeats of the tetrapeptide sequence Glx-Pro-Thr-Thr, or variants thereof, which are indicated by boxes.

FIG. 8 depicts the binding of C1INH to fluid phase P- and E-selectins. Plasma-derived C1INH (10 μg) was incubated with the E- or P-selectin IgG chimeric proteins for 60 minutes at 37° C., following which Protein G agarose was added and incubation continued for 60 minutes. After washing, proteins were eluted with sample buffer and subjected to SDS-PAGE. Western blots were probed with anti-C1INH antiserum.

FIGS. 9A-B are graphs depicting the inhibition of U937 cell transmigration across endothelial monolayers by native C1INH (FIG. 9A) and reactive center cleaved C1INH (FIG. 9B). HUVEC (3×10⁴) were seeded onto the filter and grown to a monolayer in 24-well plates, and then were stimulated with TNF-α (50 ng/ml, 18 h). The integrity of the monolayer was tested with FITC-BSA. U937 cells were labeled with BCECF-AM (5×10⁵ cells, 100 μl) in the absence or presence of either native or reactive center cleaved C1INH at the indicated concentrations and were incubated with the HUVEC at 37° C. for 45 minutes. The cells that had migrated from the upper to the lower chamber were quantitated by measurement of the fluorescence intensity at an excitation peak of 485 nm and an emission peak of 530 nm.

FIG. 10 depicts the blocking of neutrophil infiltration by C1INH in a local inflammation model. Local inflammation was induced in the skin of Balb/c mice by administration of LPS (50 ng s.c.). Immediately after injection of LPS, C1INH (300 μg i.v.), iC1INH (300 μg i.v.) or vehicle (PBS) were administered. Four hours after LPS treatment, the mice were killed and skin samples were stained with H&E.

FIG. 11 is a graph depicting the mean number of leukocytes which migrated in mice with peritonitis in response to administration of various forms of C1INH. Peritonitis was induced in mice by i.p. injection of 3% thioglycollate (0.5 ml). C1INH was administered immediately before the thioglycollate injection. Neutrophil recruitment to the peritoneal cavity was assessed at 4 hours after injection. Data are expressed as the mean number of leukocytes ±SEM. (TG, thioglycollate; iC1INH, inactive, trypsin-cleaved C1INH; dN-C1INH, deglycosylated C1INH with removal of the N-glycans.)

FIG. 12 illustrates that recombinant C1INH expressed in LEC11 cells bears more sialyl-Lewis^{x}. The recombinant C1INH expressed in LEC11 cells (LEC11-C1INH), together with plasma C1INH, was subjected to SDS-PAGE and Western blot analysis with the mAb HECA-452. The LEC11-C1INH was quantitated using ELISA and the amount of loaded proteins was as indicated.

### DETAILED DESCRIPTION OF THE INVENTION

The present invention is based, at least in part, on the discovery of a novel cell-to-cell adhesion function of C1INH that is unrelated to its previously identified protease inhibitory activity, e.g., the inhibition of the activation of the complement system through inhibition of C1, C1r, or C1s, or the inhibition of the activation of the contact system through inhibition of kallikrein, factor XIa, or factor XIIa. It has been found that C1INH contains a specific glycoprotein moiety on its N-glycan, e.g., a sialyl Lewis^{x}-related moiety, and directly interacts with, e.g., specifically binds to, selectin adhesion molecules, e.g., E-selectin (formerly ELAM-1), L-selectin (formerly LAM-1) and P-selectin (formerly PADGEM or GMP-140), including soluble P-selectin.

The expression of selectins on cells, e.g., endothelial cells, mediates the cell-to-cell adhesion, e.g., capture, adherence, migration, or “rolling,” via a selectin-specific ligand, and a sialyl-Lewis^{x} moiety of target cells, e.g., leukocytes or platelets, to the vascular wall, e.g., the endothelium or endothelial cells. Subsequent firm adherence of the leukocytes to endothelial cells is mediated by interactions between integrins and ICAM-1 and leads to transendothelial migration of leukocytes into tissue and is an essential component of a cell adhesion related disorder, e.g., an inflammatory response. In addition to their role in leukocyte-endothelial cell adhesion, leukocyte rolling and extravasation in inflammation, the expression of selectins on other cells, e.g., platelets, e.g., activated platelets, mediates the interaction between platelets and leukocytes, e.g., within thrombi. This interaction increases tissue factor expression on monocytes, thereby promoting fibrin deposition by leukocytes and thrombogeneisis (Palabrica, et al. (1992) Nature 359:848-851; Celi, A. et al. (1994) Proc Natl Acad Sci USA 91:8767-8771, the contents of which are expressly incorporated herein by reference). Furthermore, expression of soluble P-selectin induces a procoagulant state in mammals by means of an increase in the number of microparticles containing tissue factor in the blood, reducing bleeding time and/or clotting time (see U.S. application Publication No. US-2002-0031508).

It has been found that a C1INH-type protein contains a sialyl-Lewis^{x} moiety and binds to selectin molecules, e.g., P-, E-, and L-selectin, including soluble P-selectin, and modulates, e.g., inhibits or decreases, cell-to-cell adhesion and migration, e.g., endothelial-leukocyte adhesion and migration and leukocyte-platelet adhesion, and inhibits platelet and leukocyte adhesion to arterial walls. Therefore, C1INH-type protein binding to selectin molecules suppresses an immune response, e.g., an inflammatory response, and treats and prevents cell adhesion related diseases, including inflammatory diseases or disorders and thrombotic diseases or disorders. Accordingly, cell-to-cell adhesion may be modulated, e.g., inhibited, by an agonist of C1INH-type protein activity.

The terms “agonist of C1INH-type protein activity,” “C1INH-type protein agonist,” or “agonist of C1INH activity” as used interchangeably herein, include any enhancer or promoter of C1INH-type protein activity or expression. For example, a C1INH-type protein agonist may increase expression or activity of endogenous C1INH-type protein in a subject. C1INH-type protein agonists include, for example, C1INH-type proteins, e.g., C1INH-type protein, or fragments thereof, nucleic acid molecules that encode C1INH-type protein, or fragments thereof, enhancers of C1INH-type protein transcription or enhancers of C1INH-type protein translation, enhancers of post-translational modification of C1INH-type protein, including glycosylation, mimetics of C1INH-type protein such as small molecules or peptidomimetics, such as, for example, peptide fragments which mimic the binding interaction of C1INH-type proteins to selectin molecules, or variants of C1INH-type protein which mimic the binding interaction of C1INH-type proteins to selectin molecules.

C1INH participates in the down-regulation of leukocyte migration from the vasculature during an inflammatory response. During the early stages of inflammation, endothelial E-selectin and P-selectin are upregulated but C1INH levels remain normal and therefore are unlikely to interfere with leukocyte rolling. As the acute inflammatory response develops, the C1INH concentration increased up to 2.5 fold. At these, or higher concentrations, C1INH, likely with al-acid glycoprotein, as well as other selectin ligands, interferes with the leukocyte-selectin interaction, which results in the inhibition of migration of cells to inflammatory sites.

The binding of C1INH to selectin molecules on the endothelial surface may also serve to localize and concentrate C1INH at these sites, which would result in more efficient local regulation of activation of the complement and contact systems. This would further suppress vascular permeability mediated by the contact system and the inflammatory effects mediated by complement system activation.

Furthermore, inhibiting soluble P-selectin activity using a C1INH-type protein, or fragment thereof which is capable of binding P-selectin, also regulates, e.g., reduces hemostasis by binding P-selectin and decreasing the level of soluble P-selectin, which is shed from activated platelets, thereby treating or preventing thrombus formation and thrombotic diseases.

A “C1INH-type protein” includes a polypeptide, or fragment thereof which is capable of binding to or contains a sialyl-Lewis^{x} moiety, is capable of binding a selectin molecule, e.g., via a sialyl-Lewis^{x} moiety, and/or is capable of modulating cell-to-cell adhesion or cell migration, e.g., via a sialyl-Lewis^{x} moiety. In one embodiment, a C1INH-type protein includes a polypeptide which is capable of inhibiting activated components of the classical complement pathway, C1, C1r and C1s, or is capable of inhibiting the intrinsic contact system, factor XIa, factor XIIa and kallikrein. In another embodiment, a C1INH-type protein may contain an amino terminal domain which is a heavily glycosylated mucin-like domain comprising amino acids 1-120 of C1INH. In another embodiment, the N-terminal domain of a C1INH-type protein comprises amino acids 1-97 of C1INH, and contains or is capable of binding a sialyl-Lewis^{x} moiety, and/or is capable of binding a selectin molecule, e.g., via a sialyl-Lewis^{x} moiety. The domain contains up to 7 repeats of the tetrapeptide sequence Glx-Pro-Thr-Thr, or variants thereof. In another embodiment, C1INH-type protein may also contain a “serpin domain” (also referred to herein as a “serpin reactive center loop,” or a “center reactive loop”) comprising amino acid residues 98 through the C-terminus of C1INH (see Bock et al. (1986), supra), which contains or is capable of binding a sialyl-Lewis^{x} moiety and/or is capable of binding a selectin molecule, e.g., via a sialyl-Lewis^{x} moiety. An intact, e.g., functional, unmodified, serpin reactive domain is essential for protease inhibitory activity of C1INH-type proteins.

In another embodiment, a C1INH-type protein comprises the amino acid sequence set forth as SED ID NO:2, or a fragment thereof, and is encoded by the nucleotide sequence set forth in SEQ ID NO:1, or a fragment thereof (see, for example, Bock et al. (1986) Biochemistry 25:4292-4301 and Coutino, et al. (1994) J. Immunol 153:3648-3654, and GenBank Accession No. GI:179620, the contents of which are incorporated herein by reference). The methods of the invention encompass the use of nucleic acid molecules that differ from the nucleotide sequence shown in SEQ ID NO:1 due to degeneracy of the genetic code and thus encode the same C1INH proteins as those encoded by the nucleotide sequence shown in SEQ ID NO:1.

A “fragment of a C1INH-type protein” as used herein, includes a polypeptide which comprises less than the full-length polypeptide and includes a polypeptide which is capable of binding to or contains a sialyl-Lewis^{x} moiety, is capable of binding a selectin molecule, e.g., via a sialyl-Lewis^{x} moiety, and/or is capable of modulating cell-to-cell adhesion or cell migration, e.g., via a sialyl-Lewis^{x} moiety. In one embodiment, the fragment lacks an N-terminal domain. In another embodiment, the fragment lacks an intact serpin reactive center loop, referred to herein as “reactive center cleaved C1INH.” In another embodiment, the fragment comprises at least one mucin-like domain. In yet another embodiment, the fragment comprises one or more, preferably 2, 3, 4, 5, 6, or up to 7 tetrapeptide sequences. In still another embodiment, the fragment comprises amino acids 1-97 of C1INH, e.g., the N- or amino-terminal domain, or an active fragment thereof, and contains or is capable of binding a sialyl-Lewis^{x} moiety. In another embodiment, the fragment comprises amino acids 98-478 of C1INH, e.g., the C- or carboxy-terminal domain, or an active fragment thereof and contains or is capable of binding a sialyl-Lewis^{x} moiety. In another embodiment, a fragment of a C1INH-type protein comprises a portion of the C1INH polypeptide which contains or is capable of binding a sialyl-Lewis^{x} moiety. In another embodiment, a fragment of a C1INH-type protein comprises a portion of the C1INH polypeptide and is capable of binding to a selectin molecule, e.g., via a sialyl Lewis^{x} moiety, but does not function as a protease inhibitor, e.g., it does not bind or inhibit complement pathway activation, e.g., through inhibition of C1, C1r, and C1s. In still another embodiment, a fragment of a C1INH-type protein contains or is capable of binding a sialyl-Lewis^{x} moiety and is capable of binding to a selectin molecule, e.g., via a sialyl Lewis^{x} moiety, but does not inhibit the contact system activation, e.g., through inhibition of plasma kallikrein, factor XIa, or factor XIIa, for example.

In one aspect, the present invention is directed to the use of an agonist of a C1INH protein, e.g., a C1INH-type polypeptide which is capable of binding a sialyl-Lewis^{x} moiety and/or specifically binding a selectin molecule, e.g., via a sialyl-Lewis^{x} moiety, for the treatment and prevention of cell adhesion related disorders. In one embodiment, a C1INH-type polypeptide comprises the amino terminal domain of a C1INH-type protein. In another embodiment, a modified C1INH-type polypeptide contains an intact serpin reactive center loop. In another embodiment, deletion of the amino terminal 97 amino acid residues abrogates the ability of a C1INH-type polypeptide to express sialyl Lewis^{x} moiety. In another embodiment, deletion of the carboxy-terminal amino acid residues, e.g., amino acid residues 98-478 abrogates the ability of a C1INH-type polypeptide express a sialyl Lewis^{x} moiety. In another embodiment, deletion of the amino terminal 97 amino acid residues abrogates the ability of a C1INH-type polypeptide to interact with a selectin molecule. In another embodiment, deletion of the carboxy-terminal amino acid residues abrogates the ability of a C1INH-type polypeptide to interact with a selectin molecule. Moreover, in another embodiment, reactive center cleaved C1INH-type polypeptide, e.g., a C1INH-type protein which is unable to act as a protease inhibitor because it lacks an intact center reactive loop, acts to prevent or treat cell adhesion related disorders in a subject. Thus, in one embodiment, a C1INH-type polypeptide which comprises the amino terminal domain, e.g., amino acids 1-97 of C1INH, or a fragment thereof, can be used to treat or prevent a cell adhesion related disorder in a subject. In another embodiment, a C1INH-type polypeptide which comprises the carboxy-terminal domain, or a fragment thereof, can be used to treat or prevent a cell adhesion related disorder in a subject. In another embodiment, a fragment of a C1INH-type protein which comprises the amino terminal domain, e.g., amino acids 1-97 of a C1INH-type protein, can also be used to modulate selectin-mediated inflammation. In another embodiment, a fragment of a C1INH-type protein which comprises the C-terminal domain, e.g., amino acids 98-478 of a C1INH-type protein, can also be used to modulate selectin-mediated inflammation. In a further embodiment, a fragment of a C1INH-type protein which comprises the amino terminal domain, e.g., amino acids 1-97 of a C1INH-type protein, can also be used to suppress the binding of selectin expressing cells to other inflammatory mediating cells, e.g., leukocytes and platelets. In a further embodiment, a fragments of a C1INH-type protein which comprises the amino terminal domain, e.g., amino acids 98-478 of a C1INH-type protein, can also be used to suppress the binding of selectin expressing cells to other inflammatory mediating cells, e.g., leukocytes and platelets.

In another aspect, the invention provides a method for modulating the binding of a C1INH-type protein, comprising contacting a C1INH-type protein with a composition comprising an agent which specifically binds to a C1INH-type protein but does not substantially inhibit the complement system, e.g., by inhibition of C1, C1r, or C1s, thereby modulating the binding of a C1INH-type protein to a cell, e.g., leukocytes and platelets. In one embodiment, the agent does not substantially inhibit the complement system.

In still another aspect, the invention provides a method for modulating the binding of a C1INH-type protein, comprising contacting a C1INH-type protein with a composition comprising an agent which specifically binds a C1INH-type protein but does not substantially inhibit the contact system, e.g., by inhibition of kallikrein, factor XIa, or factor XIIa, thereby modulating the binding of a C1INH-type protein to a cell, e.g., leukocytes and platelets. In one embodiment, the agent does not substantially inhibit the contact system.

As used herein, the phrase “reduced or substantially eliminated protease inhibitory activity” means that the protease inhibitory activity of a protease inhibitor, e.g., a C1INH-type protein, or a fragment thereof, is reduced. That is, while there may be some protease inhibitor activity, inhibition of proteases, e.g., C1, C1r, or C1s or kallikrein, factor XIa, or factor XIIa, is not carried out to the fullest extent.

As used herein, the phrase “does not substantially inhibit activation of the complement system or contact system” means that inhibition of activation of the complement or contact system is inhibited to some extent but may not be completely inhibited. Inhibition of the activation of the complement system or the contact system can be assayed for by identifying the presence of SDS-stable enzyme-inhibitor complexes and proteolytically cleaved C1INH (see, e.g., Schapira et al. (1988) Methods Enzymol 163:179-185).

As used interchangeably herein, “C1INH-type protein activity,” “C1INH activity,” “biological activity of C1INH” or “functional activity of C1INH,” includes an activity exerted by a C1INH-type protein, polypeptide or nucleic acid molecule on a C1INH-responsive cell, e.g., platelet, leukocyte, or endothelial cell, or molecule, e.g., a selectin molecule, as determined in vivo, or in vitro, according to standard techniques. C1INH-type protein activity can be a direct activity, such as an association with a C1INH-target molecule e.g., a selectin molecule, e.g., via a sialyl Lewis^{x} moiety. As used herein, a “substrate” or “target molecule” or “binding partner” is a molecule, e.g., a selectin molecule, with which a C1INH-type protein binds or interacts in nature, such that C1INH-type protein-mediated function, e.g., modulation of cell adhesion or migration, is achieved. Alternatively, a C1INH-type protein activity is an indirect activity, such as a cellular signaling activity mediated by interaction of the C1INH-type protein with a C1INH-type protein target molecule. The biological activities of C1INH-type proteins are described herein, and include, for example, one or more of the following activities: 1) binding to or interacting with a selectin molecule, e.g., P-selectin, e.g., soluble P-selectin, E-selectin, or L-selectin, e.g., via a sialyl Lewis^{x} moiety; 2) modulating selectin binding; 2) modulating cell-to-cell adhesion, e.g., platelet-leukocyte adhesion or endothelial-leukocyte adhesion; 3) modulating cell migration, e.g., leukocyte recruitment to platelets and endothelial cells; 4) and modulating a cell adhesion related disease or disorder.

A used herein, the term “cell-to-cell adhesion” refers to adhesion between at least two cells, e.g., platelets, leukocytes, or endothelial cells, through an interaction between a selectin molecule and a selectin specific ligand, e.g., C1INH, or an active fragment thereof. Cell-to-cell adhesion includes cell migration, including leukocyte rolling.

A “cell adhesion related disorder” is defined herein as any disease or disorder which results from or is related to cell-to-cell adhesion or migration. A cell adhesion disorder also includes any disease or disorder resulting from inappropriate, aberrant or abnormal activation of the immune system or the inflammatory system. Such diseases include, without limitation, myocardial infarction, bacterial or viral infection, metastatic conditions, e.g., cancer, inflammatory disorders such as arthritis, gout, uveitis, acute respiratory distress syndrome, asthma, emphysema, delayed type hypersensitivity reaction, systemic lupus erythematosus, thermal injury such as burns or frostbite, autoimmune thyroiditis, experimental allergic encephalomyelitis, multiple sclerosis, multiple organ injury syndrome secondary to trauma, diabetes, Reynaud's syndrome, neutrophilic dermatosis (Sweet's syndrome), inflammatory bowel disease, Grave's disease, glomerulonephritis, gingivitis, periodontitis, hemolytic uremic syndrome, ulcerative colitis, Crohn's disease, necrotizing enterocolitis, granulocyte transfusion associated syndrome, cytokine-induced toxicity, fetal development, and the like.

A cell adhesion related disorder also includes thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, atherosclerosis, angina, e.g., unstable angina, disseminated intravascular coagulation, thrombotic thrombocytopenic purpura, cancer metastasis, sickle cell disease, and glomerular nephritis. In addition, inhibitors of soluble P-selectin expression or activity, e.g., a C1INH-type protein of the invention, are administered to prevent thrombotic events or to prevent re-occlusion during or after therapeutic clot lysis or procedures such as angioplasty or surgery.

Administration of an agonist of C1INH-type protein activity, e.g., a C1INH-type protein, or a fragment thereof, or a nucleic acid molecule encoding a C1INH type protein, or a fragment thereof, to a subject for the treatment or prevention of a cell adhesion related disorder may be alone or in combination with other agents known to aid in the treatment or prevention of cell adhesion related disorders, e.g., antihistamines or anti-inflammatory agents. In another embodiment, when therapeutically beneficial, an agonist of C1INH-type protein activity may be administered in combination with any agent which acts as a protease inhibitor to inhibit the complement system, e.g., through inhibition of C1, C1s, or C1r, and/or any agent which inhibits contact system activation, e.g., through inhibition of plasma kallikrein, factor XIa, or factor XIIa, for example. Administration of an agonist of C1INH -type protein activity and another agent may be serialy or as a mixture.

Isolated C1INH-type protein, purified from cells or recombinantly produced, may be used as a pharmaceutical composition when combined with a pharmaceutically acceptable carrier. Such a composition may contain, in addition to C1INH-type protein and carrier, diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration.

Various aspects of the invention are described in further detail in the following subsections:

I. Methods of Treatment of Subjects Suffering from a Cell Adhesion Related Disorder

The present invention provides for both prophylactic and therapeutic methods of treating a subject, e.g., a human, at risk of (or susceptible to) a cell adhesion related disorder. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with an agonist of C1INH-type protein activity, for example, a C1INH-type protein or fragment thereof, or a nucleic acid molecule encoding a C1INH type protein, a fragment thereof, or C1INH-type protein modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing a cell adhesion related disorder in a subject by administering to the subject an agonist of C1INH-type protein activity, e.g., an enhancer of C1INH transcription or translation, e.g., endogenous C1INH transcription or translation. In one embodiment, the present invention provides methods for preventing a cell adhesion related disorder in a subject by administering to the subject a C1INH-type protein, or a fragment thereof which contains or is capable of binding a sialyl LewisX related moiety and/or is capable of binding a selectin molecule, e.g., E-selectin, P-selectin, including soluble P-selectin, or L-selectin. In another aspect, the invention provides a method for preventing a cell adhesion related disorder in a subject by administering to the subject a nucleic acid molecule which encodes a C1INH-type protein, or a fragment thereof which contains or is capable of binding sialyl Lewis^{x} related moiety and/or is capable of binding a selectin molecule, e.g., E-selectin, P-selectin, including soluble P-selectin, or L-selectin. Subjects at risk for cell adhesion related disorders can be identified by, for example, any or a combination of the diagnostic or prognostic assays described herein. Administration of a prophylactic agent can occur prior to the manifestation of symptoms characteristic of cell adhesion related disorder, e.g., prior to manifestation of disease or infection which places a subject at risk for a cell adhesion related disorder, such that a cell adhesion related disorder is prevented or, alternatively, delayed in its progression.

B. Therapeutic Methods

Another aspect of the invention pertains to methods for treating a subject suffering from a cell adhesion related disorder. These methods involve administering to a subject an agonist of C1INH-type protein activity, e.g., an enhancer of C1INH transcription or translation, or post-transcriptional modification, e.g., glycosylation, a C1INH-type protein, or fragment thereof, or a C1INH-type protein mimetic, e.g., a small molecule, or a nucleic acid molecule which encodes a C1INH-type protein, or a fragment thereof, as therapy for a cell adhesion related disorder. Administration of an agonist of C1INH-type protein activity, e.g., a C1INH-type protein or nucleic acid molecule encoding C1INH, or a fragment thereof, to a subject for the treatment or prevention of a cell adhesion related disorder may be alone or in combination with other agents known to aid in the treatment or prevention of a cell adhesion related disorder, e.g., antihistamines, anti-inflammatory agents. In another embodiment, when therapeutically beneficial, an agonist of C1INH-type protein activity may be administered in combination with any agent which acts as a protease inhibitor to inhibit the complement system, e.g., through inhibition of C1, C1s, or C1r, and/or any agent which inhibits contact system activation, e.g., through inhibition of plasma kallikrein, factor XIa, or factor XIIa, for example. Administration of an agonist of C1INH-type protein activity and another agent may be serially or as a mixture.

An agonist of C1INH-type protein activity, e.g., a C1INH-type protein, nucleic acid molecule encoding C1INH, or a fragment thereof, can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use, include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating C1INH-type protein or a fragment thereof, or C1INH-type nucleic acid molecule encoding C1INH, or a fragment thereof, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

An agonist of C1INH-type protein activity, e.g., a C1INH-type protein or a fragment thereof, or C1INH-type nucleic acid molecule encoding C1INH, or a fragment thereof, can also be prepared in the form of suppositories (e.g., with conventional suppository bases such as cocoa butter and other glycerides) or retention enemas for rectal delivery.

In one embodiment, an agonist of C1INH-type protein activity, e.g., a C1INH-type protein or a fragment thereof, or C1INH-type nucleic acid molecule encoding C1INH, or a fragment thereof, is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates C1INH-type protein activity and the particular therapeutic effect to be achieved, and the limitations inherent in the art of compounding such an agent for the treatment of subjects.

Toxicity and therapeutic efficacy of an agonist of C1INH-type protein activity, e.g., a C1INH-type protein or a fragment thereof, or C1INH-type nucleic acid molecule encoding C1INH, or a fragment thereof, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents that exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such C1INH-type protein or a fragment thereof lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured; for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which mimic C1INH-type protein selectin binding activity, e.g., an agonist of C1INH-type protein activity. An agonist may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agonist of C1INH-type protein activity, e.g., a C1INH-type protein or a fragment thereof, or C1INH-type nucleic acid molecule encoding C1INH, or a fragment thereof, as well as tailoring the dosage and/or therapeutic regimen of treatment with an agonist of C1INH-type protein activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known, all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., C1INH-type protein or a fragment thereof, or a mimetic, or a nucleic acid molecule encoding a C1INH-type protein or a fragment thereof) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cell adhesion related disorder with an agonist of C1INH-type protein activity, e.g., a C1INH-type protein or a fragment thereof, or C1INH-type nucleic acid molecule encoding C1INH, or a fragment thereof.

II. Screening Assays:

The invention provides methods (also referred to herein as “screening assays”) for identifying modulators, i.e., candidate or test compounds or agents (e.g., peptides, peptidomimetics, small molecules, ribozymes, or C1INH-type protein antisense molecules) which have an inhibitory effect on the activity of a C1INH-type protein target ligand, e.g., E-selectin, P-selectin, including soluble P-selectin, or L-selectin. The invention also provides methods (also referred to herein as “screening assays”) for identifying modulators, i.e., agonists of C1INH-type protein activity (e.g., peptides, peptidomimetics, small molecules, enhancers of C1INH transcription or translation, or post-transcriptional modification, e.g., glycosylation) which increase or promote C1INH-type protein expression or activity, e.g., endogenous C1INH expression or activity. Compounds identified using the assays described herein may be useful for treating cell adhesion related disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L- configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay that may be used to identify compounds that modulate C1INH-type protein activity is a cell-based assay in which a cell which expresses a C1INH-type protein or biologically active portion thereof (e.g., the portion of C1INH-type protein that binds to a selectin molecule) of the C1INH-type protein that is necessary for specific binding to a selectin molecule, is contacted with a test compound and the ability of the test compound to modulate C1INH-type protein activity is determined. Determining the ability of the test compound to modulate C1INH-type protein activity can be accomplished by monitoring, for example, C1INH-type protein binding to selectin molecules, e.g., E- and P-selectin, cell-to-cell adhesion, e.g., endothelial-leukocyte or platelet-leukocyte binding, C1INH-type protein binding to soluble P-selectin, C1INH-type protein or other inflammatory mediators, or direct binding of modified C1INH-type protein, or a fragment thereof, to selectin molecules, as described herein. Other assays known in the art or described herein may be used to determine the ability of a test compound to modulate C1INH-type protein activity, e.g., binding to selectin expressing cells or soluble selectin molecules.

The ability of the test compound to modulate C1INH-type protein binding to selectin molecules can also be determined. Determining the ability of the test compound to modulate C1INH-type protein binding to selectin molecules can be accomplished, for example, by coupling C1INH-type protein with a radioisotope or enzymatic label such that binding of selectin molecules to C1INH-type protein can be determined by detecting the labeled C1INH-type protein in a complex. Alternatively, C1INH-type protein could be coupled with a radioisotope or enzymatic label to monitor the ability of a test compound to modulate C1INH-type protein binding to a selectin molecule in a complex. Determining the ability of the test compound to bind C1INH-type protein can be accomplished, for example, by coupling the compound with a radioisotope or enzymatic label such that binding of the compound to C1INH-type protein can be determined by detecting the labeled C1INH-type protein compound in a complex. For example, C1INH-type protein substrates can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with C1INH-type protein without the labeling of any of the interactants. For example, a microphysiometer can be used to detect the interaction of a compound with C1INH-type protein without the labeling of either the compound or the C1INH-type protein (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and C1INH-type protein.

The ability of a C1INH-type protein modulator to modulate, e.g., inhibit or increase, C1INH-type protein activity can also be determined through screening assays which identify modulators which either increase or decrease binding of C1INH-type protein or a fragment thereof to selecting, e.g., E-, P-selectin or soluble P-selectin. In one embodiment, the invention provides for a screening assay involving contacting cells which express a C1INH-type protein or a fragment thereof with a test compound and a selectin molecule, and measuring the binding of C1INH-type protein or a fragment thereof, to a selectin molecule, via, e.g., methods described herein.

To determine whether a test compound modulates C1INH-type protein expression, in vitro transcriptional assays can be performed. To perform such an assay, the full length promoter and enhancer of C1INH-type protein can be linked to a reporter gene such as chloramphenicol acetyltransferase (CAT) and introduced into host cells. The same host cells can then be transfected with the test compound. The effect of the test compound can be measured by testing CAT activity and comparing it to CAT activity in cells which do not contain the test compound. An increase or decrease in CAT activity indicates a modulation of C1INH-type protein expression and is, therefore, an indicator of the ability of the test compound to bind to selectin molecules.

In yet another embodiment, an assay of the present invention is a cell-free assay in which C1INH-type protein or biologically active portion thereof (e.g., the portion of C1INH-type protein that is involved in the binding to selectin molecules) is contacted with a test compound and the ability of the test compound to bind to or to modulate (e.g., stimulate or inhibit) the activity of C1INH-type protein or biologically active portion thereof is determined. Preferred biologically active portions of C1INH-type proteins to be used in assays of the present invention include fragments which are capable of specifically binding selectin molecules, e.g., fragments comprising amino acids 1-97 of C1INH, fragments comprising the C-terminal amino acids 98-478, or a fragment thereof. Determining the ability of C1INH-type protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize either C1INH-type protein or selectins to facilitate separation of complexed from uncomplexed forms of one or both of the proteins, as well as to accommodate automation of the assay. Binding of a test compound to a C1INH-type protein, or interaction of a C1INH-type protein with selectins in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/C1INH-type protein fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or C1INH-type protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of C1INH-type binding or activity determined using standard techniques.

Other techniques for immobilizing proteins on matrices can also be used in the screening assays of the invention. For example, either C1INH-type protein or selectin molecules can be immobilized utilizing conjugation of biotin and streptavidin. Biotinylated C1INH-type protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with C1INH-type protein or target molecules but which do not interfere with binding of the C1INH-type protein to its target molecule can be derivatized to the wells of the plate, and unbound target or C1INH-type protein is trapped in the wells by antibody conjugation. Methods for detecting such complexes, in addition to those described above for the GST-immobilized complexes, include immunodetection of complexes using antibodies reactive with the C1INH-type protein or selectin molecules, as well as enzyme-linked assays which rely on detecting an enzymatic activity associated with the C1INH-type protein and selectin molecules.

In yet another aspect of the invention, the C1INH-type protein or fragments thereof (e.g., the portion of C1INH-type protein that is involved in the binding to selectin molecules) can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with C1INH-type proteins (“C1INH-type-binding proteins” or “C1INH-type-bp) and are involved in C1INH-type protein activity. Such modified C1INH-type-binding proteins are also likely to be involved in the propagation of signals by the C1INH-type proteins as, for example, downstream elements of a C1INH-type protein-mediated signaling pathway. Alternatively, such C1INH-type-binding proteins are likely to be C1INH-type protein inhibitors.

The two-hybrid system is based on the modular nature of most transcription factors, which consist of separable DNA-binding and activation domains. Briefly, the assay utilizes two different DNA constructs. In one construct, the gene that codes for a C1INH-type protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a C1INH-type protein-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the C1INH-type protein.

In another aspect, the invention pertains to a combination of two or more of the assays described herein. For example, a modulating agent can be identified using a cell-based or a cell-free assay, and the ability of the agent to modulate the activity of a C1INH-type protein can be confirmed in vivo, e.g., in an animal, such as an animal model for inflammation. Examples of animals that can be used include animals, e.g., mice, rabbits, or baboons, which have been administered, e.g., topically applied, injected or inhaled, an agent that induces an immune response, e.g., ovalbumin, sodium lauryl sulfate, or thioglycolate, in the animal, as described in, for example, Hopken, U E, et al., (1997) J Exp Med, 186:749-56; Melnicoff, M. J, et al., (2002) Toxicol Appl Pharmacol, 182:126-35; Horan, P. K. and P. S. Morahan (1989) Cell Immunol, 118:178.

Moreover, a modulator, e.g., agonist, of C1INH activity identified as described herein can be used in an animal model to determine the efficacy, toxicity, or side effects of treatment with such a modulator. Alternatively, a modulator, e.g., agonist, of C1INH activity identified as described herein can be used in an animal model to determine the mechanism of action of such a modulator.

III. Isolated Nucleic Acid Molecules of the Invention

The coding sequence of the isolated human C1INH-type protein cDNA and the predicted amino acid sequence of the human C1INH-type polypeptide are shown in SEQ ID NOs:1 and 2, respectively. The C1INH sequence is also described in Bolk, et al. (1986), Biochemistry 25:4292-4301.

The C1INH-type protein nucleic acid molecules of the invention includes an isolated nucleic acid molecule that encodes a C1INH-type protein, e.g., or a fragment thereof, which contains a sialyl-Lewis^{x} moiety, and/or is capable of binding a selectin molecule, e.g., via a sialyl-Lewis^{x} moiety. In one embodiment, the isolated nucleic acid molecules encode a polypeptide comprising amino acids 1-97 of C1INH (SEQ ID NO:2), or a fragment thereof which is capable of specifically binding selectin molecules, e.g., via a sialyl-Lewis^{x} moiety. In another embodiment, the isolated nucleic acid molecules encode a polypeptide comprising amino acids 98-478 of C1INH (SEQ ID NO:2), or a fragment thereof which is capable of specifically binding selectin molecules, e.g., via a sialyl-Lewis^{x} moiety.

In another embodiment, the isolated nucleic acid molecules are nucleic acid fragments sufficient for use as hybridization probes to identify C1INH-type protein-encoding nucleic acid molecules (e.g., C1INH-type protein mRNA) and fragments for use as PCR primers for the amplification or mutation of C1INH-type protein nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a fragment thereof, can be isolated using standard molecular biology techniques and the sequence information provided herein. Using all or a portion of the nucleic acid sequence of SEQ ID NO:1 as a hybridization probe, C1INH-type protein nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, or a fragment thereof, can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to C1INH-type protein nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:2 or a portion of any of this nucleotide sequence, e.g., a portion encoding the amino terminal domain of C1INH-type protein.

Moreover, the nucleic acid molecules used in the methods of the invention can comprise only a portion of the nucleic acid sequence of SEQ ID NO:1, for example, a fragment which can be used as a probe or primer or a fragment encoding a portion of a C1INH-type protein, e.g., a biologically active portion of a C1INH-type protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, of an anti-sense sequence of SEQ ID NO:1 or of a naturally occurring allelic variant or mutant of SEQ ID NO:1. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, or more nucleotides in length and hybridizes under stringent hybridization conditions to a nucleic acid molecule of SEQ ID NO:2.

As used herein, the term “hybridizes under stringent conditions” is intended to describe conditions for hybridization and washing under which nucleotide sequences which are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(logio[Na⁺])+0.41(%G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a C1INH-type protein, such as by measuring a level of a C1INH-type protein-encoding nucleic acid in a sample of cells from a subject e.g., detecting C1INH-type protein mRNA levels or determining whether a genomic C1INH-type protein gene has been mutated or deleted.

The methods of the invention further encompass the use of nucleic acid molecules that differ from the nucleotide sequence shown in SEQ ID NO:1 due to degeneracy of the genetic code and thus encode the same C1INH-type proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human C1INH-type protein, e.g., fictional and non-functional allelic variants. Functional allelic variants are naturally occurring amino acid sequence variants of the human C1INH-type protein that maintain a C1INH-type protein activity, e.g., the ability to bind selectin molecules. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human C1INH-type protein that do not have a C1INH-type protein activity, e.g., the ability to bind selectin molecules. Non-functional allelic variants will typically contain a non-conservative substitution, deletion, or insertion or premature truncation of the amino acid sequence of SEQ ID NO:2, or a substitution, insertion or deletion in critical residues or critical regions of the protein.

The methods of the present invention may further use non-human orthologues of the human C1INH-type protein. Orthologues of the human C1INH-type protein are proteins that are isolated from non-human organisms and possess the same C1INH-type protein activity.

Particular modified C1INH-type polypeptides which can be made as described herein include C1INH-type polypeptides containing mutations which result in reduced protease inhibitory activity of the modified C1INH-type protein. For example, disruption or cleavage of the serpin center reactive loop domain of C1INH-type protein can result in modified C1INH-type polypeptides which have reduced protease activity but retain the ability to specifically bind to selectin molecules. Furthermore, modified, e.g., truncated C1INH-type polypeptides which result from the cleavage of amino acids 98-478, or a portion thereof, retain selectin binding activity but have reduced protease inhibitory activity.

The methods of the present invention further include the use of nucleic acid molecules comprising the nucleotide sequence of SEQ ID NO:1 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of C1INH-type protein (e.g., the sequence of SEQ ID NO:2) without altering the biological activity, e.g., specific binding to selectin molecules, whereas an “essential” amino acid residue is required for biological activity. For example, amino acid residues that are conserved among the C1INH-type proteins of the present invention and other members of the protease inhibitor family, those amino acid residues and domains that contain or express a sialyl Lewis^{x} moiety, and those amino acid residues that bind selectin molecules, e.g., via a sialyl Lewis^{x} moiety, are not likely to be amenable to alteration.

Mutations can be introduced into SEQ ID NO:2 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a C1INH-type protein is preferably replaced with another amino acid residue from the same side chain family. Alternatively, in another embodiment, mutations can be introduced randomly along all or part of a C1INH-type protein coding sequence, such as by saturation mutagenesis, and the resultant mutants can be screened for C1INH-type protein biological activity to identify mutants that retain activity, e.g., specific binding to selectin molecules. Following mutagenesis of SEQ ID NO:1 the encoded protein can be expressed recombinantly and the activity of the protein can be determined using the assays described herein.

Another aspect of the invention pertains to the use of isolated nucleic acid molecules which are antisense to the nucleotide sequence of SEQ ID NO:1, or fragments thereof. An “antisense” nucleic acid comprises a nucleotide sequence which is complementary to a “sense” nucleic acid encoding a protein, e.g., complementary to the coding strand of a double-stranded cDNA molecule or complementary to a mRNA sequence. Accordingly, an antisense nucleic acid can hydrogen bond to a sense nucleic acid. The antisense nucleic acid can be complementary to an entire C1INH-type protein coding strand, or to only a portion thereof. In one embodiment, an antisense nucleic acid molecule is antisense to a “coding region” of the coding strand of a nucleotide sequence encoding a C1INH-type protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding C1INH-type protein. The term “noncoding region” refers to 5′ and 3′ sequences which flank the coding region that are not translated into amino acids (also referred to as 5′ and 3′ untranslated regions).

Given the coding strand sequences encoding C1INH-type protein disclosed herein, antisense nucleic acids of the invention can be designed according to the rules of Watson and Crick base pairing. The antisense nucleic acid molecule can be complementary to the entire coding region of C1INH-type protein mRNA, but more preferably is an oligonucleotide which is antisense to only a portion of the coding or noncoding region of C1INH-type protein mRNA. For example, the antisense oligonucleotide can be complementary to the region surrounding the translation start site of C1INH-type protein mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouraci 1, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a C1INH-type protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave C1INH-type protein mRNA transcripts to thereby inhibit translation of C1INH-type protein mRNA. A ribozyme having specificity for a C1INH-type protein-encoding nucleic acid can be designed based upon the nucleotide sequence of a C1INH-type protein cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a C1INH-type protein -encoding mRNA. See, e.g., Cech et al. U.S. Pat. No. 4,987,071; and Cech et al. U.S. Pat. No. 5,116,742. Alternatively, C1INH-type protein mRNA can be used to select a catalytic RNA having a specific ribonuclease activity from a pool of RNA molecules. See, e.g., Bartel, D. and Szostak, J. W. (1993) Science 261:1411-1418.

Alternatively, C1INH-type protein gene expression can be inhibited by targeting nucleotide sequences complementary to the regulatory region of C1INH-type protein (e.g., the C1INH-type protein promoter and/or enhancers) to form triple helical structures that prevent transcription of the C1INH-type protein gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12):807-15.

In yet another embodiment, the C1INH-type protein nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of C1INH-type protein nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of C1INH-type protein nucleic acid molecules can also be used in the analysis of single base pair mutations in a gene (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of C1INH-type protein can be modified, (e.g., to enhance their stability), by attaching lipophilic or other helper groups to PNA, by the formation of PNA-DNA chimeras, or by the use of liposomes or other techniques of drug delivery known in the art. For example, PNA-DNA chimeras of C1INH-type protein nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

IV. Isolated C1INH-Type Proteins of the Invention

The invention includes isolated C1INH-type proteins, and fragments thereof, e.g., C1INH-type polypeptides which are capable of binding a selectin molecule, e.g., via a sialyl-Lewis^{x} moiety. In one embodiment, the invention includes isolated polypeptides of C1INH-type protein, or a fragment thereof which is capable of specifically binding a selectin molecule, e.g., via a sialyl-Lewis^{x} moiety. The invention also includes polypeptide fragments suitable for use as immunogens to raise anti-C1INH-type protein antibodies. In one embodiment, native C1INH-type proteins can be isolated from cells or tissue sources by an appropriate purification scheme using standard protein purification techniques. In another embodiment, C1INH-type proteins are produced by recombinant DNA techniques. Alternative to recombinant expression, a C1INH-type protein or polypeptide can be synthesized chemically using standard peptide synthesis techniques.

As used herein, a “biologically active portion” of a C1INH-type protein includes a fragment of a C1INH-type protein having a C1INH-type activity, e.g., the ability to bind selectins, e.g., via a sialyl-Lewis^{x} moiety. Biologically active portions of a C1INH-type protein include peptides comprising amino acid sequences sufficiently identical to or derived from the amino acid sequence of the C1INH protein, e.g., the amino acid sequence shown in SEQ ID NO:2, which include fewer amino acids than the full length C1INH-type proteins, and exhibit at least one activity of a C1INH-type protein, e.g., specific binding to selecting. Typically, biologically active portions comprise a domain or motif with at least one activity of the C1INH-type protein (e.g., the amino-terminal domain of the C1INH-type protein, the serpin domain). A biologically active portion of a C1INH-type protein can be a polypeptide which is, for example, 5, 10, 15, 20, 25, 30, 35, 40, 45, 50, 55, 60, 65, 70, 75, 80, 85, 90, 95, or 100 or more amino acids in length. Biologically active portions of a C1INH-type protein can be used as targets for developing agents which modulate a C1INH-type protein activity, e.g., binding to selecting.

In a preferred embodiment, the C1INH-type protein used in the methods of the invention has an amino acid sequence shown in SEQ ID NO:2, or a fragment thereof. In other embodiments, the C1INH-type protein is substantially identical to SEQ ID NO:2, or a fragment thereof, and retains the functional activity of the protein of SEQ ID NO:2, yet differs in amino acid sequence due to natural allelic variation or mutagenesis, as described in detail in subsection III above. Accordingly, in another embodiment, the C1INH-type protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, or 50%, 55%, 60%, 65%, 70%, 75%, 80%, 85%, 90%, 95%, 96%, 97%, 98%, 99% or more identical to a fragment of C1INH-type protein.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the C1INH-type protein amino acid sequence of SEQ ID NO:2 having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at the Accelrys™ website), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at the Accelrys™ website), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM 120 weight residue table, a gap length penalty of 12 and a gap penalty of 4.

The methods of the invention may also use C1INH-type protein chimeric or fusion proteins. As used herein, a C1INH-type protein “chimeric protein” or “fusion protein” comprises a C1INH-type polypeptide, or a fragment thereof, operatively linked to a non-C1INH-type polypeptide. A “C1INH-type polypeptide” refers to a polypeptide having an amino acid sequence corresponding to a C1INH-type protein molecule, or a fragment thereof, whereas a “non-C1INH-type polypeptide” refers to a polypeptide having an amino acid sequence corresponding to a protein which is not substantially homologous to the C1INH-type protein, or a fragment thereof, e.g., a protein which is different from the C1INH-type protein and which is derived from the same or a different organism. Within a C1INH-type protein fusion protein the C1INH-type polypeptide, or a fragment thereof, can correspond to all or a portion of a C1INH-type protein. In a preferred embodiment, a C1INH-type protein fusion protein comprises at least one biologically active portion of a C1INH-type protein, e.g., the amino terminal domain or a fragment thereof or the serpin domain or a fragment thereof. In another preferred embodiment, a C1INH-type protein fusion protein comprises at least two biologically active portions of C1INH-type protein. Within the fusion protein, the term “operatively linked” is intended to indicate that the C1INH-type polypeptide and the non-C1INH-type polypeptide are fused in-frame to each other. The non-C1INH-type polypeptide can be fused to the N-terminus or C-terminus of the C1INH-type polypeptide, or a fragment thereof.

For example, in one embodiment, the fusion protein is a GST-C1INH-type protein fusion protein in which the C1INH-type protein sequences are fused to the C-terminus of the GST sequences. Such fusion proteins can facilitate the purification of recombinant C1INH-type protein.

In another embodiment, this fusion protein is a C1INH-type protein containing a heterologous signal sequence at its N-terminus. In certain host cells (e.g., mammalian host cells), expression and/or secretion of C1INH-type protein can be increased through use of a heterologous signal sequence.

The C1INH-type protein fusion proteins used in the methods of the invention can be incorporated into pharmaceutical compositions and administered to a subject in vivo. The C1INH-type protein fusion proteins can be used to affect the bioavailability of selectin molecules. Moreover, the C1INH-type protein-fusion proteins used in the methods of the invention can be used as immunogens to produce anti-C1INH-type protein antibodies in a subject, to purify C1INH-type protein ligands and in screening assays to identify molecules which inhibit the interaction of C1INH-type proteins with a C1INH-type protein substrate.

Preferably, a C1INH-type protein chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A C1INH-type protein-encoding nucleic acid can be cloned into such an expression vector such that the fusion moiety is linked in-frame to the C1INH-type protein.

The present invention also pertains to the use of variants of the C1INH-type proteins which function as C1INH-type protein agonists (mimetics). Variants of the C1INH-type proteins can be generated by mutagenesis, e.g., discrete point mutation or truncation of a C1INH-type protein. An agonist of the C1INH-type proteins can retain substantially the same, or a subset, of the biological activities of the naturally occurring form of a C1INH-type protein, e.g., the ability to bind selecting. Thus, specific biological effects can be elicited by treatment with a variant of limited function.

In one embodiment, variants of a C1INH-type protein which function as C1INH-type protein agonists (mimetics) can be identified by screening combinatorial libraries of mutants, e.g., truncation mutants, of a C1INH-type protein for C1INH-type protein agonist activity. In one embodiment, a variegated library of C1INH-type protein variants is generated by combinatorial mutagenesis at the nucleic acid level and is encoded by a variegated gene library. A variegated library of C1INH-type protein variants can be produced by, for example, enzymatically ligating a mixture of synthetic oligonucleotides into gene sequences such that a degenerate set of potential C1INH-type protein sequences is expressible as individual polypeptides, or alternatively, as a set of larger fusion proteins (e.g., for phage display) containing the set of C1INH-type protein sequences therein. There are a variety of methods which can be used to produce libraries of potential C1INH-type protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential C1INH-type protein sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a C1INH-type protein coding sequence can be used to generate a variegated population of C1INH-type protein fragments for screening and subsequent selection of variants of a C1INH-type protein. In one embodiment, a library of coding sequence fragments can be generated by treating a double stranded PCR fragment of a C1INH-type protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the C1INH-type protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of C1INH-type protein. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify C1INH-type protein variants (Arkin and Yourvan (1992) Proc. Natl. Acad. Sci. USA 89:7811-7815; Delgrave et al. (1993) Protein Engineering 6(3):327-331).

V. Recombinant Expression Vectors and Host Cells Used in the Methods of the Invention

The methods of the invention (e.g., the screening assays described herein) include the use of vectors, preferably expression vectors, containing a nucleic acid encoding C1INH-type protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., C1INH-type proteins, mutant forms of C1INH-type proteins, fragments of C1INH-type proteins, fusion proteins, and the like).

The recombinant expression vectors to be used in the methods of the invention can be designed for expression of C1INH-type proteins in prokaryotic or eukaryotic cells. For example, C1INH-type proteins can be expressed in bacterial cells, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in C1INH-type protein activity assays, (e.g., direct assays or competitive assays described in detail herein), or to generate antibodies specific for C1INH-type proteins.

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to C1INH-type protein mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a C1INH-type protein nucleic acid molecule of the invention is introduced, e.g., a C1INH-type protein nucleic acid molecule within a recombinant expression vector or a C1INH-type protein nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A number of types of cells may act as suitable host cells for expression of the C1INH-type proteins of the invention. Suitable host cells are capable of attaching carbohydrate side chains characteristic of functional C1INH-type proteins. Such capability may arise by virtue of the presence of a suitable glycosylating enzyme within the host cell, whether naturally occurring, induced by chemical mutagenesis, or through transfection of the host cell with a suitable expression plasmid containing a DNA sequence encoding the glycosylating enzyme, e.g., a fucosyltransferase. Host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, or HaK cells. Other suitable host cells are known to those skilled in the art.

The C1INH-type proteins of the invention may also be produced by operably linking the isolated DNA of the invention and one or more DNAs encoding suitable glycosylating enzymes to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference.

Alternatively, it may be possible to produce the C1INH-type proteins of the invention in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the C1INH-type proteins of the invention is made in yeast or bacteria, it is necessary to attach the appropriate carbohydrates to the appropriate sites on the protein moiety covalently, in order to obtain the glycosylated C1INH-type proteins of the invention. Such covalent attachments may be accomplished using known chemical or enzymatic methods.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a C1INH-type protein. Accordingly, the invention further provides methods for producing a C1INH-type protein using the host cells of the invention. In one embodiment, the method comprises culturing the host cell of the invention (into which a recombinant expression vector encoding a C1INH-type protein has been introduced) in a suitable medium such that a C1INH-type protein is produced. In another embodiment, the method further comprises isolating a C1INH-type protein from the medium or the host cell. In certain preferred embodiments, C1INH-type protein is produced by co-transfecting a host cell with DNA encoding a C1INH-type protein and a DNA encoding a fucosyltransferase capable of synthesizing sialyl Lewis X (sLe^{x}) or sialyl Lewis A (sLe^{a}) (such as an (α1,3/α1,4) fucosyltransferase or an (α1,3) fucosyltransferase).

This invention is further illustrated by the following examples which should not be construed as limiting. The contents of all references, patent applications, patents, and published patent applications, as well as the Figures and the Sequence Listing cited throughout this application are hereby incorporated by reference.

### EXAMPLES

Materials and Methods

The following materials and methods were used for the experiments described below.

Plasmids and Protein

The plasmids encoding human P- or E-selectin-IgG chimeric proteins are described in Aruffo, A., et al. (1991) Cell 67:35 and Bevilacqua, M. P., et al. (1989) Science 243:160. The selectin portion of these two constructs includes the signal sequence, the lectin domain, the epidermal growth factor-like repeat, and the first two consensus repeats fused to the hinge region followed by the CH2 and CH3 domain of human IgG₁. Plasma C1 inhibitor and C1s were obtained from Advanced Research Technologies (San Diego, Calif.). Recombinant E-selectin was purchased from Calbiochem, EMD Biosciences, Inc (San Diego, Calif.).

Cell-Lines

The cell line PRO^{—}LEC11.E7 (generic name LEC11) is described in Campbell, C., et al., (1984) J Biol Chem 259:11208. The cell line is a Chinese hamster ovary (CHO) cell mutant that has an active α(1,3)-fucosyltransferase that can add fucose to certain sialylated glycoproteins and makes possible the biosynthesis of the sialyl Lewis^{x} tetrasaccharide during post-translational glycosylation. LEC11 was cultured in alpha MEM (Invitrogen, Carlsbad, Calif.). All other cell lines were from ATCC (American Type Culture Collection, Rockville, Md.) and cultured according to ATCC protocols. These included CHO-K1, the human monocytic cell line U937, and human umbilical vein endothelial cells (HUVEC).

Antibodies

Rabbit anti-human C1INH antiserum was from DAKO (Denmark), and mouse anti-human P- and E-selectin mAbs were from BD Pharmingen (San Diego, Calif.). Peroxidase-conjugated secondary antibodies against rabbit IgG, mouse IgG, rat IgM, and mouse IgM were from Pierce (Rockford, Ill.).

The mAb HECA-452 and CSLEX1 producing hybridomas were cultured in RPMI-1640. The mAb, HECA452, a rat IgM, can recognize sialyl Lewis^{x} related carbohydrate ligands (sialyl Lewis^{x} and its isoform sialyl Lewis^{a}) for human E-selectin, including the T-cell E-selectin ligand cutaneous lymphocyte antigen (Picker, L. J., et al. J. Immunol. 145:3247; Duijvestijn, A. M., et al. (1988) Am. J. Pathol. 130:147; Berg, E. L., et al. (1991) J. Exp. Med. 174:1461). The mAb CSLEX1 is a mouse IgM that can recognize sialyl Lewis^{x} (Fukushima, K. M., et al., (1984) Cancer Res 44:5279). Both antibodies have been used extensively in identification of selectin ligands (Fukushima, K. M., et al., (1984) Cancer Res 44:5279; Tu, L., et al., (1999) J Exp Med 189:241; Zollner, O., et al., (1996) J Biol Chem 271:33002).

Expression and Purification of P, E-Selectin-IgG Chimeric Protein

P- and E-selectin-IgG chimeric plasmids were co-transfected into CHO-K1 cells with pcDNA3.1. The clones with the highest expression level were selected with G418 sulfate (1 mg/ml), and the resulting stable cell lines were named CHO/P and CHO/E, respectively. P and E-selectin expression was confirmed by Western blot analysis using mAbs against human P or E-selectin. P- and E-selectin were purified from the culture medium using Protein G-agarose and eluted with 4 M imidazole.

Expression and Purification of Recombinant C1INH

The full-length C1INH construct including the signal sequence, the coding region and partial transcriptional initial and terminal sequences in the expression vector pcDNA3.1 (−) (Coutinho, M., et al., (1994) J Immunol 153:3648), was used to transfect CHO-K1 and LEC11 cells, respectively. The high-expressing clones were selected in growth medium containing G418 (1 mg/ml).

After centrifugation at 15000 g for 30 min to remove cell debris, the conditioned medium was concentrated using a Centricon Plus-80 (Millpore, Bedford, Mass.) and diluted with PBS, pH7.4 containing 10 mM EDTA, 25 μM p-nitrophenyl-p-guanidino benzoate and 1 mM PMSF, and applied to a jacalin-agarose (Vector, Burlingame, Calif.) column, which was pre-equilibrated with the same buffer. After washing with 10-column volumes of the starting buffer containing 0.5 M NaCl, C1INH was eluted with 10-column volume of 0.125 M melibiose in the same buffer. The C1INH pool from the jacalin-agraose column was concentrated, (NH₄)₂SO₄ was added to a final concentration of 0.4M and applied to a phenyl-Sepharsoe column in a ÄKTA FPLC system (Amersham, Piscataway, N.J.). The flowthrough, containing C1INH was collected and thereafter changed to PBS, pH7.4 using a desalting column. C1INH concentration was determined by ELISA (Coutinho, M., et al., (1994) J Immunol 153:3648).

Fluorescence-Activated Cell Sorting (FACS)

CHO/P, CHO/E and un-transfected CHO-K1 cells (1×10⁶) were trypsinized and washed with PBS. Cells were incubated with human plasma-derived C1INH at 250 μg/ml in PBS containing 1 mM MgCl₂ and 1 mM CaCl₂ at 37° C. for 60 minutes. After washing three times with the same buffer, cells were incubated with rabbit anti-human C1INH antiserum (1/100 dilution) at 37° C. for 60 minutes and washed as above. Cells then were incubated with goat anti-rabbit IgG-fluorescein isothiocyanate (FITC) (Caltag laboratories, Burlingame, Calif.) (1:1000 dilution of 0.8 mg/ml) at 37° C. for 60 minutes and washed 5 times. Cells were analyzed on a FACScan instrument using CellQuest software (Becton Dickinson Immunocytometry Systems, San Jose, Calif.).

Deglycosylation

To determine whether HECA-452 epitopes on C1INH were dependent on sialic acid and/or O-linked sialoglycoproteins, C1INH (20 μg) was incubated with 2.5 mL of O-glycosidase and neuraminidase (Roche, Germany) or 5 U of N-glycosidase F (New England Biolab, Mass.) at 37° C. overnight in a buffer containing 50 mM sodium phosphate, pH7.5 and 1% NP-40. Deglycosylated C1INH was subjected to Western blot analysis as described below.

Western Blot

In order to determine whether C1INH bears sialyl Lewis^{x} related moieties, C1INH, ranging from 1 to 8 μg was separated on 6% SDS-PAGE. BSA (20 μg) and CHO-K1 lysate (1×10⁶ cells) were included as negative controls, while LEC11 and U937 lysate (1×10⁶ cells) were used as positive controls. Proteins were transferred onto a nitrocellulose membrane. After blocking with PBS containing 0.05%Tween-20 and 5% non-fat milk, the blot was probed with mAb HECA-452 or CSLEX1 (concentrated conditioned culture medium). Blots were stripped with 0.2 N NaOH, blocked and reprobed with anti-C1INH antiserum. Secondary antibodies were HRP-conjugated goat anti-rat IgM (1/5,000 dilution), anti-mouse IgM, or anti-rabbit IgG (1/10,000 dilution), respectively. The proteins were detected with a SuperSignal Chemiluminescent Substrate kit (Pierce, Rockford, Ill.) and signals were developed using X-OMAT AR film (Eastman Kodak, Rochester, N.Y.).

To determine if N-linked glycosylation contributed to the HECA-452 reactive epitope on C1INH, O- and N-glycosidase treated-plasma-derived C1INH was subjected to SDS-PAGE and probed with HECA-452 and anti-C1INH antiserum, respectively, as described above.

In order to determine if the HECA452-reactivity of C1INH is defined by a sialyl LewisX moiety as a result of the presence of active α1,3-fucosyltransferase, recombinant C1INH from LEC11 and CHO-K1 cells was separated by SDS-PAGE, blotted and probed with HECA-452. The same blot, after stripping, was reprobed with anti-C1INH antiserum, as described above.

Complex-Formation Assay

A C1INH-C1s complex-formation assay was used to determine whether the C1INH-selectin interaction interfered with the proteinase inhibitory function of C1INH. C1INH (2 μl of 1 mg/ml) was incubated with or without E-selectin (2 μl of 1 mg/ml) at 37° C. for 60 minutes in PBS containing 1 mM CaCl₂ and 1 mM MgCl₂. C1s (2 μl of 1 mg/ml) was added and incubation continued at 37° C. for 60 minutes. Samples then were subjected to SDS-PAGE and stained with Coommassie blue.

Immunoprecipitation

HUVEC cells (at <10 early passage), stimulated with TNF-α and H₂O₂ as described below, were incubated with C1INH (125 μg/ml) at 37° C. for 60min, and then lysed directly in the tissue-culture flask (2 ml for a 75-cm² flask) with lysis buffer (1% Brij97, 10 mM Tris-HCl, pH 7.4, 150 mM NaCl, 1 mM CaCl₂, 1 MM MgCl₂, 1 mM PMSF, 25 μM p-nitrophenyl-p-guanidino benzoate). After incubation for 45 minutes at 4° C., insoluble material was removed by centrifugation at 10,000 g. Thereafter, all steps are performed at 4° C. The cell lysate was precleared overnight by addition of preimmune rabbit serum and Protein G-agarose beads (Sigma, Saint Louis, Mo.). Proteins then were immunoprecipitated from the supernatant with rabbit anti-human C1INH antiserum (20 μl per ml) and Protein G-agarose beads (20 μl per ml). After incubation for 5 hrs under constant agitation, the beads were washed five times in lysis buffer. The bound proteins were eluted with EDTA and subjected to SDS-PAGE and Western blot analysis with mAb against P or E-selectin, respectively.

Endothelial-Leukocyte Adhesion Assay

HUVEC (under 10 generations) were plated into 96-well flat-bottom fibronectin-coated plates (BD Bioscience) at 3×10⁴ cells per well 2 days before the assay. The cells were treated with human TNF-α (50 ng/ml) for 4 hrs and with H₂O₂ (250 μM) for 5 mins at 37° C. Human plasma-derived C1INH in 0.5×HUVEC culture medium was added at the indicated concentration and incubated for 1 hr at 37° C. A control containing EDTA (1 mM) and a control without C1INH added were included. The human monocytic cell line U937 was labeled by adding 10 μM of 2′,7′-bis-(2-carboxyethyl)-5-(and-6)-carboxyfluorescein, acetoxymethyl ester (BCECF-AM) (Molecular probes, Eugene, Oreg.) and incubated at 37° C. for 30 minutes. Labeled cells (100 μl of 5×10⁶) were added into each well and incubated for 45 minutes at 37° C. Cells then were washed with HUVEC culture medium by gentle swirling, followed by inverting the plate and blotting the unbound cells 4 times. PBS (100 μl) containing BSA (100 μg/ml) was added into each well and the fluorescence was measured using a fluorescence reader (Dynex Technologies, Chantilly, Va.) at an excitation peak of 485 nm and an emission peak of 530 nm.

### Example 1

### C1INH Bears a Sialyl Lewis^{x}-Related Moeity on its N-Linked Carbohydrate

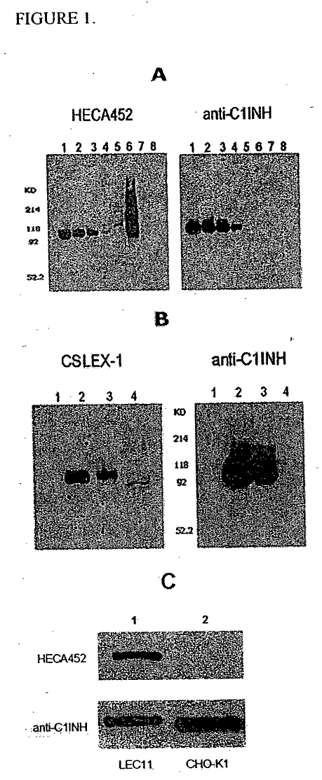

To determine if plasma C1INH expresses a sialyl Lewis^{x}-related moiety, reactivity of C1INH with mAbs HECA-452 and CSLEX1 was tested. Western blot analysis indicated that plasma C1INH bears a HECA-452 and a CSLEX1-reactive epitope (FIGS. 1A, B). While untransfected CHO-K1 cells and BSA show no signal, cell lysates from U937 and LEC11 cells show distinct reactivity. The differences in reactivity of the two mAbs with U937 cells are consistent with previous observations (Zak, I. E., et al. (2000) Acta Biochim 47:393).

To test if the HECA-452 reactivity of C1INH is specifically determined by the sialyl Lewis^{x} moiety, the synthesis of which is catalyzed by α(1,3)-fucosyltransferase, C1INH was expressed in LEC11 and CHO-K1 cells. The isolated recombinant protein from the LEC11 cells was detected with HECA452. This protein also reacted with anti-C1INH antiserum. However, the recombinant protein from CHO-K1 cells did not react with HECA452 (FIG. 1C). These results indicate that the HECA452 reactivity of C1INH is dependent on the α(1,3)-fucosyltransferase that is present in LEC11 cells but absent in CHO-K1 cells.

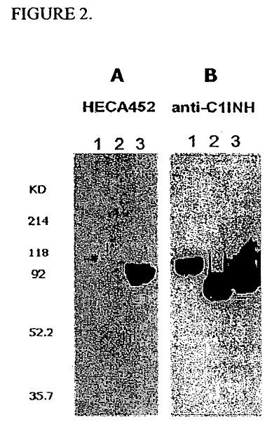

Plasma C1INH contains both N- and O-glycans. To determine whether N or O-linked carbohydrate contributed the HECA reactivity, C1INH was treated with O-glycosidase and N-glycosidase F. Deglycosylation was confirmed by the size decrease on SDS-PAGE (FIG. 2). C1INH deglycosylated with N-glycosidase F lost its HECA-reactivity (FIG. 2), which indicates that the sialyl Lewis^{x}-related moiety is located on the N-glycan of C1INH.

### Example 2

### C1INH Binds to P- and E-Selectin Adhesion Molecules

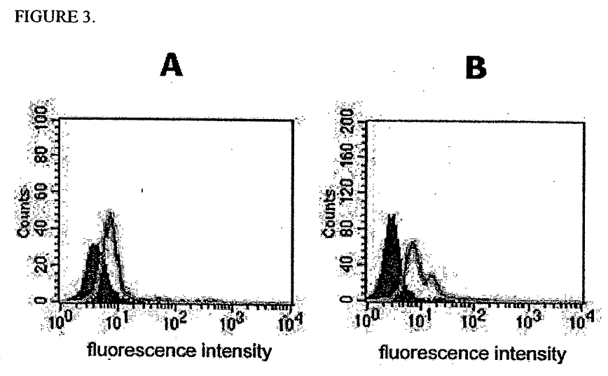

FACS analysis showed that plasma-derived C1INH can bind P- and E-selectin adhesion molecules expressed as the P- or E-selectin/IgG chimera on the transfected cell surface (FIG. 3). CHO/P, CHO/E and untransfected CHO-K1 cells were incubated with human C1 inhibitor (250 μg/ml) at 37° C. for 60 minutes. The bound C1INH was detected with rabbit anti-human C1INH antiserum and FITC-conjugated goat anti-rabbit IgG. The binding of C1INH to the P- and E-selectin/IgG chimeric molecules is similar to that of the soluble complement receptor I modified to express the sialyl Lewis^{x}-related moiety in LEC11 cells (Ritterhaus, C. W., et al., (1999) J Biol Chem 274:11237). It is also consistent with the observation that binding of selectins and their ligands characteristically is of low affinity. It has been hypothesized that low affinity binding is a general feature of cell adhesion receptors (van der Merwe, P. A. and Barclay, A. N. (1994) Trends Biochem Sci 19:354).

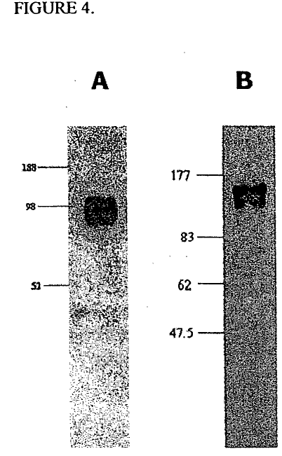

To further investigate whether circulating C1INH binds to P- and/or E-selectins on the surface of endothelial cells, expression of P- and E-selectin on HUVEC was upregulated by treatment with TNF-α and H₂O₂, following which the cells were incubated with human C1INH. C1INH subsequently was immunoprecipitated with rabbit anti-human C1INH antiserum. The bound proteins were separated on SDS-PAGE and Western blots were probed for P- and E-selectin. The results show that P- and E-selectin are co-immunoprecipitated with C1INH (FIG. 4). This indicates that C1INH binds to P- and E-selectin on the surface of the endothelial cell.

### Example 3

### C1INH, in the Presence of Selectin Adhesion Molecules, Retains its Full Protease Inhibitory Function

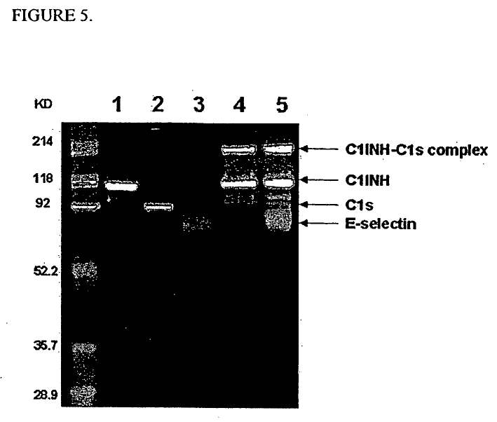

C1INH, like other serpins, forms a SDS-resistant complex with target proteases. The protease within this complex is inactivated. Complex-formation assays, therefore, can provide information about the protease inhibitory capacity of C1INH (Coutinho, M., et al., (1994) J Immunol 153:3648). C1INH (2 μl of 1 mg/ml) was incubated with or without E-selectin (2 μl of 1 mg/ml) at 37° C. for 60 minutes in PBS containing 1 mM CaCl2 and 1 mM MgCl2. C1s (2 μl of 1 mg/ml) was added and incubated at 37° C. for an additional 60 minutes. Samples then were subjected to SDS-PAGE and stained with Coommassie blue. As shown in FIG. 5, C1INH in the presence of E-selectin retains its ability to complex with C1s. Therefore, E-selectin does not appear to have any significant effect on the protease inhibitory function of C1INH.

### Example 4

### C1INH Inhibits Leukocyte-Endothelial Cell Adhesion

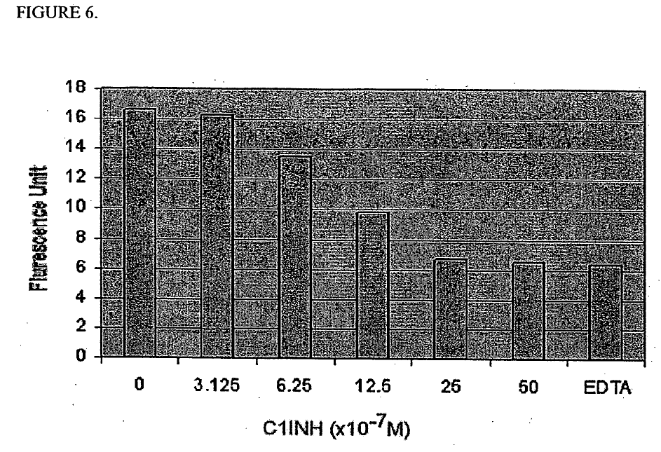

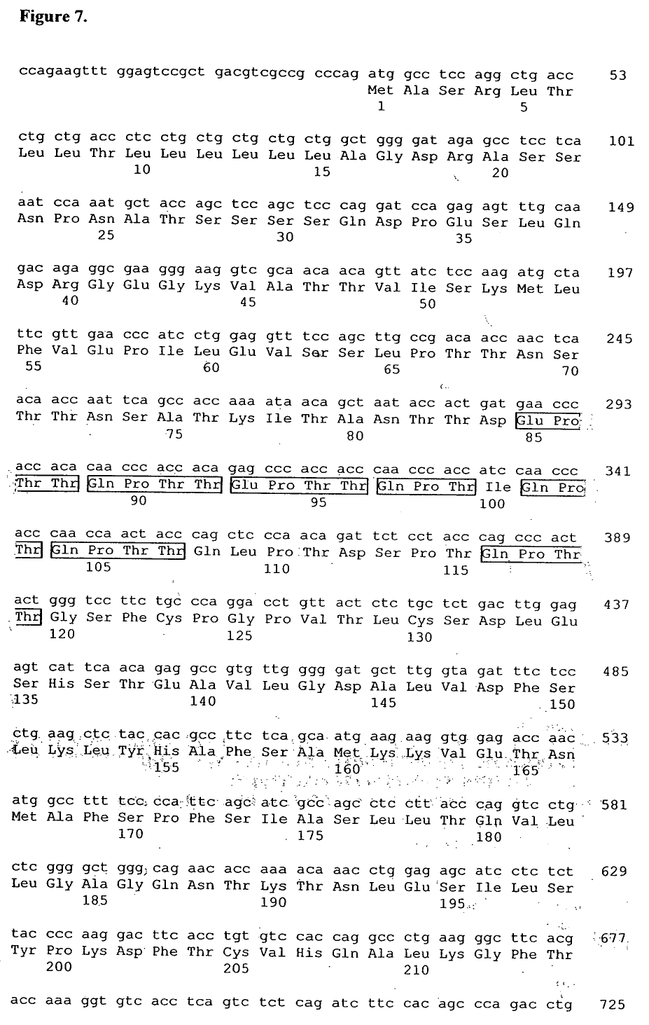

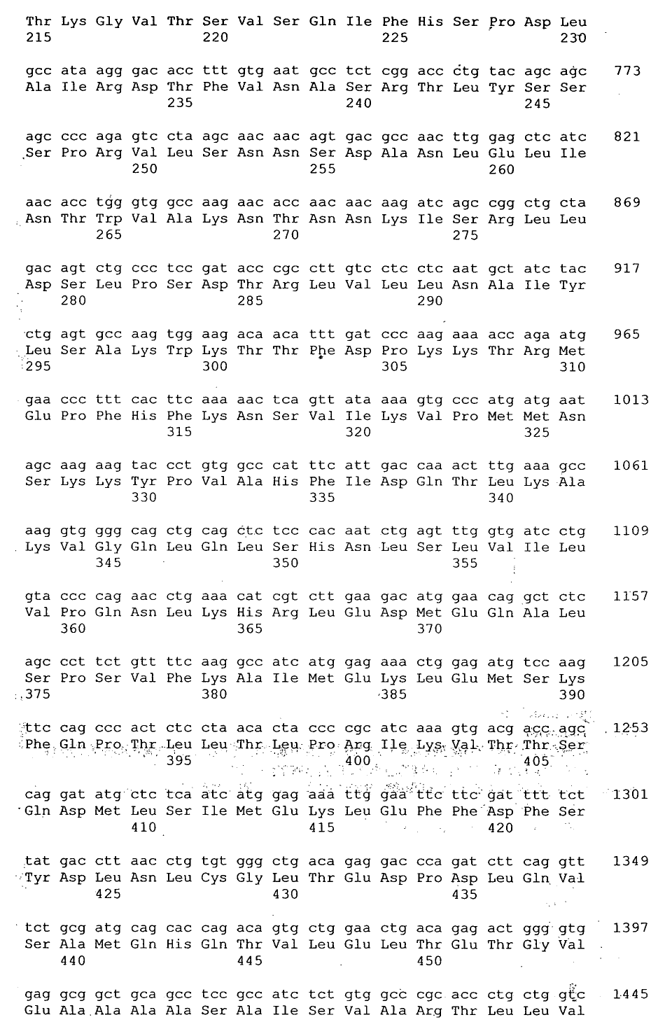

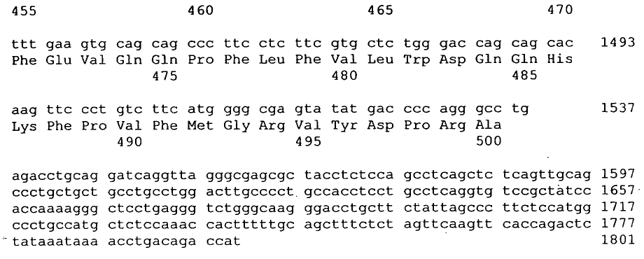

In order to determine whether C1INH can inhibit leukocyte-endothelial cell adhesion under static conditions, HUVEC were coated onto a flat-bottom fibronectin-coated plate, treated with human TNF-α and H₂O₂ and incubated with plasma C1INH. The BCECF-AM labeled U937 cells were added and incubated at 37° C. for 60 minutes. The bound cells were analyzed for fluorescence intensity at an excitation peak of 485 nm and an emission peak of 530 nm. The adhesion of fluorescent-labeled U937 cells to HUVEC was inhibited by C1INH in a dose-dependent manner (FIG. 6). The experiment was repeated 3 times and a representative result is shown in FIG. 6. Significant inhibition (>50% inhibition compared with the control) can be achieved with 250 μg/ml of C1INH, which is within the concentration range of circulating C1INH during inflammation.

### Example 5

### Binding of C1INH In Vivo

This example illustrates that C1INH competes with leukocytes for selectin binding in vivo and that this competition suppresses the migration of leukocytes from the vascular space. Three different animal models are analyzed: the Arthus reaction, sodium lauryl sulfate-induced cutaneous inflammation, and thioglylcolate-induced peritonitis. In both the Arthus reaction and thioglycolate-induced peritonitis, leukocyte migration is dependent on E- and P-selectins. The ability of intravenous C1INH to interfere with leukocyte migration is determined in both C1INH^{+/+} and C1INH^{-/-} mice in these models. Positive controls consist of mice treated with monoclonal antibodies to E- and P-selectin, which interfere with leukocyte migration. The relative roles of protease inhibition versus competitive binding to E- and P-selectin are evaluated by treating mice with mutated C1INH that has no protease inhibitor function or with intact protease inhibitor function but lacking the ability to bind to selectins.

Similar studies ultimately are performed in more complex disease models in which C1INH has been shown to be beneficial, such as reperfusion injury, hyperacute transplant rejection and respiratory distress syndrome.

### Example 6

### C1 Inhibitor Binds to Fluid Phase P- and E-Selectin

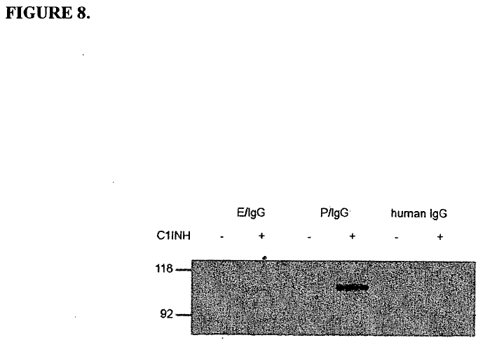

To investigate whether plasma C1INH binds to fluid phase P- and E-selectin, C1INH was incubated with fluid-phase P selectin/IgG or E selectin/IgG chimeric proteins (Aruffo, et al. (1991) Cell 67:35; Bevilacqua, et al. (1989) Science 243:160). The chimeric proteins together with any bound C1INH were precipitated with protein G-agarose. Western blot analysis of this material clearly demonstrated the presence of C1INH (FIG. 8). The specificity of the binding was confirmed by the demonstration that human IgG alone does not bind C1INH.

### Example 7

### C1INH Blocks Leukocyte-Endothelial Cell Adhesion Under Flow Conditions

The interference of C1INH with the interaction of leukocytes with E-selectin under flow conditions was assessed using an in vitro flow chamber, as described (Kadono, et al. (2002) J Immunol. 169(8):4542-50). The purified recombinant human E-selectin expressed in CHO cells (EMD Biosciences, San Diego, Calif.) in PBS, pH 9.0 at a concentration of 2 μg/ml was coated onto a 25-mm circular petri dish at room temperature for 1 hour and preincubated with 2% BSA in PBS, pH 7.0 for 1 hour to block non-specific binding. HL-60 cells, a human promyelocytic line, obtained from ATCC (Manassas, Va.), were suspended in PBS containing 1 mM CaCl2, 1 mM MgCl2, and 0.5% (w/v) BSA, at 107 cells/ml in the absence or presence of various forms of C1INH at a concentration of 300 μg/ml, and then perfused through the chamber for 20 minutes. Medium flow through the chamber was established at a calculated shear stress of 1.85 dyne/cm2 using a syringe pump (Harvard Apparatus, Natick, Mass.). After each perfusion using different forms of C1INH, the chamber was flushed first with EDTA (10 mM) to remove any attached cells, and then with PBS/BSA, pH7.0, for 5 minutes. The same coating area was examined through all perfusions. Cell rolling was observed using an inverted phase contrast microscope (Olympus, Lake Success, N.Y.) and was videotaped using a CCD video camera (Hitachi Denshi, Tokyo, Japan) with a SuperVHS video recorder (model SVO-9500 MD; Sony, New York, N.Y.) and an attached time-date generator (Microimage Video Sales, Bechtelsville, Pa.). In addition to an EDTA control, blocking monoclonal antibody against human E-selectin (clone 68-5H11, BD Biosciences Pharmingen, San Diego, Calif.) was used as a control. An uncoated area on the same dish was used as another control. The velocities of 30 rolling cells in each treatment were measured by calculating the distance traveled divided by the elapsed time (10 seconds).

Under flow conditions, a portion of the leukocytes roll on the immobilized E-selectin. This system mimics the first step in leukocyte adhesion during acute inflammation; shear strength is similar to that of a post-capillary venule. The specificity of the system was confirmed by reversal of rolling with EDTA treatment and by inhibition with the monoclonal antibody 68-5H11, which is specifically directed against human E-selectin. In addition, no rolling was observed on the uncoated area of the dish. Treatment with C1INH at a concentration similar to those observed during acute inflammation (300 μg/ml), reduced the leukocyte rolling speed by approximately 50% after treatment for 15 minutes. N-deglycosylated C1INH lost the ability to inhibit rolling.

### Example 8

### C1INH Blocks Leukocyte Trans-Endothelial Monolayer Migration In Vitro (Transwell)

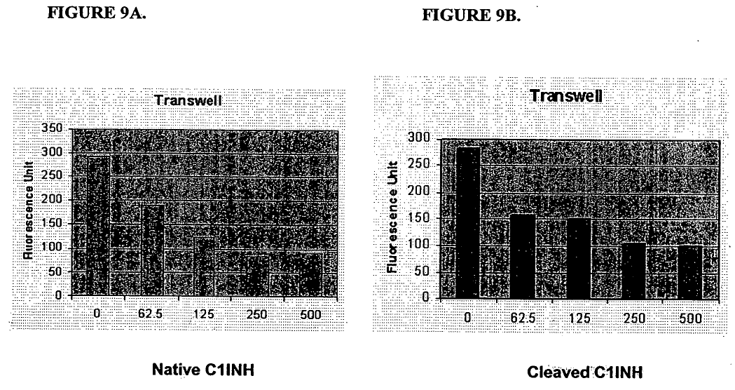

The effect of C1INH on leukocyte migration across a HUVEC monolayer was investigated using a Transwell system that is used widely as an in vitro model of leukocyte trans-endothelial infiltration (Smith, et al. (1989) J Clin Invest. 83(6):2008-17; Schenk, et al. (2002) J. Immunol. 169(5):2602-10). The endothelial cells were cultured on a filter that separates the upper and lower chambers. The permeability of the endothelial monolayer was stimulated by treatment with TNF-α. U937 cells (a human histiocytic lymphoma cell line) were added to the upper chamber and their movement into the lower chamber was quantitated. As shown in FIG. 9, C1INH inhibited TNF-α-induced migration of U937 cells across the endothelial monolayer in a dose-dependent manner. Importantly, both native C1INH and reactive center cleaved C1INH expressed this activity. Therefore, inhibition of leukocyte migration by C1INH in this system does not require protease inhibitory activity.

### Example 9

### C1INH Blocks Leukocyte Infiltration in a Local Inflammation Model

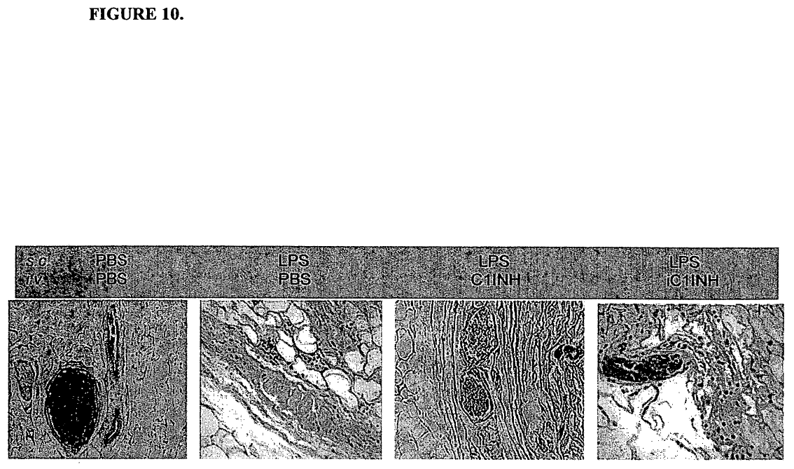

Local inflammation was induced by s.c. injection of LPS (50 μg per mouse) (Schleiffenbaum, et al. (1998) J Immunol. 161(7):3631-8) immediately after C1INH infusion (200 μg per mouse, i.v.) in Balb/c mice. PBS injection was included as a control. Skin samples (1 cm diameter) were harvested 4 hours post-injection. The section was stained with H&E for examination of neutrophil recruitment. In this endotoxin LPS-induced local inflammation model, both native and reactive center cleaved C1INH (300 μg i.v.) inhibited leukocyte infiltration into the sites of inflammation (FIG. 10).

### Example 10

### C1INH Blocks Leukocyte Infiltration in Thioglycollate Peritonitis

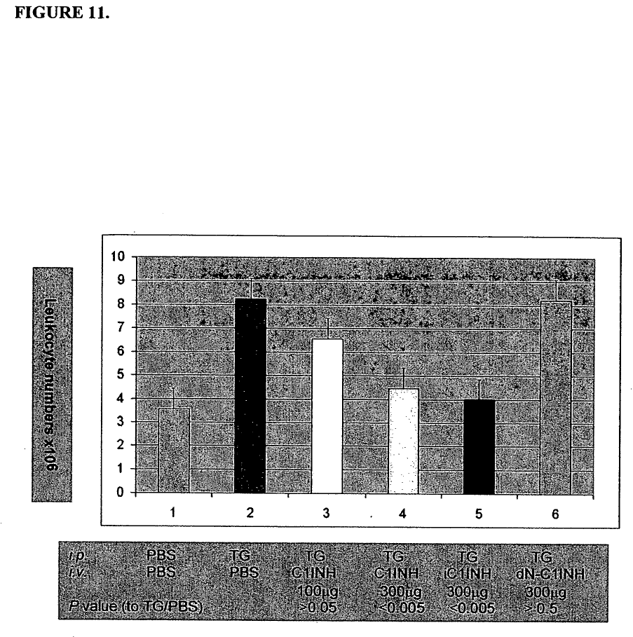

Thioglycollate is a potent reagent to induce leukocyte (mainly neutrophil) infiltration into the mouse peritoneal cavity. Thioglycollate peritonitis is a widely used model to investigate leukocyte recruitment. Mice were injected intraperitoneally with 3% thioglycollate broth (0.5 ml) (Sigma) immediately after C1INH infusion (5 or 15 mg/kg, i.v.). At 4 hours post-injection, the mice were euthanized by CO2 inhalation and peritoneal exudate cells harvested using one intraperitoneal wash with HBSS (4 ml) containing 10% FCS. Peritoneal exudate cells were counted using a Coulter Counter and stained with Wright-Giemsa stain. In this thioglycollate-induced peritonitis model, both native C1INH and reactive center cleaved C1INH inhibited thioglycollate induced leukocyte infiltration into the peritoneal cavity while N-deglycosylated C1INH lost such activity (FIG. 11). This result is consistent with the interpretation that this activity is dependent on the sialyl-Lewis^{x} moiety which is located on one or more of the N-linked carbohydrates of C1INH.

### Example 11

### C1INH Suppresses TNF-α-Induced Leukocyte Rolling In Vivo

The effect of C1INH on leukocyte rolling in vivo was examined using intravital microscopy (Mayadas, et al. (1993) Cell 74(3):541-54). TNF-α (0.5 μg, i.p., EMD Biosciences, San Diego, Calif.) was administrated 4 hours before leukocyte rolling was evaluated. The mesentery was exteriorized through a midline abdominal incision in anesthetized mice. A venule of 25 - 35 μm was located and observed for the entire procedure with a Zeiss IM35 inverted microscope connected to a SVHS video recorder (Panasonic AG-6720A, Matsushita Electric, Japan) using a CCD video camera (Hamamatsu Photonic Systems, Hamamatsu City, Japan). Exposed tissue was kept moist by periodic superfusion using PBS warmed to 37° C. Rolling leukocytes were quantitated by counting the number of cells passing a given plane perpendicular to the vessel axis in 1 minutes. Baseline rolling was determined during the first 10 minutes after surgery by taking a minimum of four 1 minute counts. Mice then were injected intravenously with the various forms of C1INH (300 μg per mouse) and changes in leukocyte rolling were quantitated over the subsequent 5-20 minutes.

The preliminary data demonstrate that both purified human C1INH (Advanced Research Technologies, San Diego, Calif.) and a human C1INH preparation, Berinert (Aventis, Strasbourg), used for replacement therapy in hereditary angioedema dramatically suppressed leukocyte rolling induced by TNF-α. Prior to treatment with TNF-α, few leukocytes are observed rolling on the endothelium. TNF-α administration (0.5 μg, i.p.) induces systemic inflammation, and at 4 hours post-treatment the rolling leukocyte numbers increase up to 30 fold. Administration of either native or reactive center cleaved, inactive C1INH dramatically reduced both the number of rolling leukocytes (by as much as 75%) and the rolling velocities. Administration of N-deglycosylated C1INH had virtually no effect on leukocyte rolling. These data demonstrate that the activity of C1INH in blocking TNF-α induced leukocyte rolling is independent of its protease-inhibitory function and suggest that it is dependent on the sialyl-Lewis^{x} moieties on its N-glycans.

Notably, in this model, C1INH was administered 4 hours after TNF-α treatment when leukocyte rolling was already induced. This mimics accurately the situation in acute inflammation. Data from this model may indicate that C1INH not only could be used as a preventive agent, but also as a therapeutic agent for a variety of inflammatory processes.

### Example 12

### Recombinant C1INH Expressed in LEC11 Cells

As noted before, C1INH expressed in LEC11 cells express the sialyl-Lewis^{x} tetrasaccharide. In order to increase the expression level and avoid loss of expression during passage, we selected transfected LEC11 cells with stable high level expression of C1INH. Full-length human C1INH cDNA was cloned into the mammalian expression vector pLXIN (Clontech). The resulting construct was transfected into LEC11 cells using electroporation. The clones with high expression were selected using puromycin (10 μg/ml). Cells were cultured in roller bottles. In seven days, the C1INH expression level was approximately 3-6 μg/ml as measured by ELISA. The purification of recombinant C1INH was achieved using jacalin-affinity, Q-Sepharose and phenyl-Sepharose chromatography as described (Davis, et al. (1993) Methods Enzymol. 223:97-120).

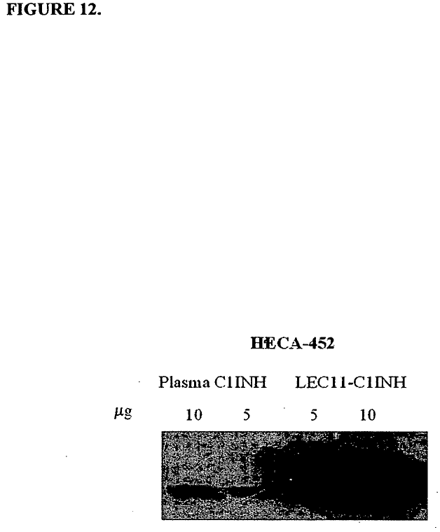

The recombinant C1INH retains protease inhibitor activity similar to that of the plasma-derived protein, as shown by its ability to form an SDS-stable complex with C1s. The presence of sialyl-Lewis^{x} on the resulting recombinant C1INH was confirmed by Western blot analysis using the HECA452 monoclonal antibody. The reactivity to HECA-452 of the recombinant C1 INH expressed in LEC11 cells is greater than that of plasma C1INH which suggests that this recombinant C1INH expresses more sialyl-Lewis^{x} than does the plasma C1INH (FIG. 12).

What is claimed:

1. A method for modulating cell-to-cell adhesion comprising contacting a cell with an agonist of C1INH-type protein activity, such that cell-to-cell adhesion is modulated.

2. The method of claim 1, wherein said cell expresses a selectin molecule selected from the group consisting of a P-selectin molecule and an E-selectin molecule.

3. The method of claim 1, wherein said agonist of C1INH-type protein activity is a C1INH-type protein, or a fragment thereof.

4. The method of claim 1, wherein said agonist of C1INH-type protein activity is C1INH, or a fragment thereof.

5. A method for modulating cell-to-cell adhesion, comprising contacting soluble P-selectin with a C1INH-type protein, such that said cell-to-cell adhesion is modulated.

6. The method of claim 5, wherein said C1INH-type protein is C1INH, or a fragment thereof.

7. The method of claim 1, wherein said cell-to-cell adhesion is decreased.

8. The method of claim 3, wherein said C1INH-type protein binds to a selectin molecule.

9. The method of claim 3, wherein said C1INH-type protein comprises the N-terminal domain of C1INH, or a fragment thereof, which is capable of binding a selectin molecule.

10. The method of claim 3, wherein said C1INH-type protein comprises the terminal domain of C1INH, or a fragment thereof, which is capable of binding a lectin molecule.

11. The method of claim 3, wherein said C1INH-type protein specifically binds a selectin molecule but does not inhibit activation of the complement system.

12. The method of claim 3, wherein said C1INH-type protein specifically binds a selectin molecule but does not inhibit activation of the contact system.

13. The method of claim 3, wherein said C1INH-type protein specifically binds a selectin molecule but has substantially reduced protease inhibition activity.

14. The method of claim 1, wherein said cell is a platelet.

15. The method of claim 1, wherein said cell is an endothelial cell.

16. The method of claim 1, wherein said cell is within a subject.

17. The method of claim 1, wherein said cell is in vitro.

18. The method of claim 16, wherein said subject is a mammal.

19. The method of claim 16, wherein said mammal is a human.

20. The method of claim 16, wherein said subject is suffering from a cell adhesion related disorder.

21. A method for treating or preventing a cell adhesion related disorder in a subject, comprising administering to said subject an effective amount of a composition comprising an agonist of C1INH-type protein activity such that said cell adhesion related disorder is treated.

22. The method of claim 21, wherein said cell adhesion related disorder is selected from the group consisting of myocardial infarction, bacterial or viral infection, metastatic conditions, arthritis, gout, uveitis, acute respiratory distress syndrome, asthma, emphysema, delayed type hypersensitivity reaction, systemic lupus erythematosus, thermal injury such as burns or frostbite, autoimmune thyroiditis, experimental allergic encephalomyelitis, multiple sclerosis, multiple organ injury syndrome secondary to trauma, diabetes, Reynaud's syndrome, neutrophilic dermatosis (Sweet's syndrome), inflammatory bowel disease, Grave's disease, glomerulonephritis, gingivitis, periodontitis, hemolytic uremic syndrome, ulcerative colitis, Crohn's disease, necrotizing enterocolitis, granulocyte transfusion associated syndrome, cytokine-induced toxicity, fetal development, or a thrombotic disorder.

23. The method of claim 22, wherein said composition further comprises a pharmaceutically acceptable carrier.

24. A method for identifying a compound capable of modulating cell to cell adhesion comprising assaying the ability of the compound to modulate C1INH-type protein activity, thereby identifying a compound capable of modulating cell to cell adhesion.

25. The method of claim 24, wherein the ability of the compound to modulate C1INH-type protein activity is determined by detecting a decrease in cell-to-cell adhesion.

26. The method of claim 24, wherein said cellular adhesion involves leukocytes, platelets, and/or endothelial cells.


SEQUENCE LISTING (2 sequences)

SEQ ID NO: 1 - DNA, 1801 bp, Homo sapiens
ccagaagtttggagtccgctgacgtcgccgcccagatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctgggcagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctctagttcaagttcaccagactctataaataaaacctgacagaccat

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA